(19) United States
(12) Patent Application Publication
(10) Pub. No.: US 2009/0291479 A1
(43) Pub. Date: Nov. 26, 2009

(54) MANIPULATION OF ACYL-COA BINDING PROTEIN EXPRESSION FOR ALTERED LIPID PRODUCTION IN MICROBIAL HOSTS

(75) Inventors: Seung-Pyo Hong, Hockessin, DE (US); Zhixiong Xue, Chadds Ford, PA (US)

Correspondence Address: E I DU PONT DE NEMOURS AND COMPANY;LEGAL PATENT RECORDS CENTER, BARLEY MILL PLAZA 25/1122B, 4417 LANCASTER PIKE, WILMINGTON, DE, 19805, US

(73) Assignee: E. I. DU PONT DE NEMOURS AND COMPANY, Wilmington, DE (US)

(21) Appl. No.: 12/469,026

(22) Filed: May 20, 2009

Related U.S. Application Data
(60) Provisional application No. 61/055,511, filed on May 23, 2008.

(51) Int. Cl. C12P 7/64; C12N 1/15; C12N 1/19

(52) U.S. Cl. 435/134; 435/254.1; 435/255.4; 435/255.3

(57) ABSTRACT

Acyl-CoA binding protein [“ACBP”] binds thiol esters of long fatty acids and coenzyme A in a one-to-one binding mode with high specificity and affinity. This protein is expected to play an important role in altering lipid production in oleaginous microbial organisms. Knock-out of the protein in the oleaginous yeast, Yarrowia lipolytica, results in a decrease in the total lipid content, while overexpression results in an increase in the total lipid content of the recombinant Yarrowia cells.

This application claims the benefit of U.S. Provisional Application No. 61/055,511, filed May 23, 2008, the contents of which are hereby incorporated by reference.

### FIELD OF THE INVENTION

This invention is in the field of biotechnology and, in particular, concerns an acyl-CoA binding protein [“ACBP”] and its use to manipulate lipid content in oleaginous microbial organisms.

### BACKGROUND OF THE INVENTION

A variety of different hosts including plants, algae, fungi, stramenopiles and yeast are being investigated as means for commercial polyunsaturated fatty acid [“PUFA”] production. As technology has developed, there is increasing emphasis on the ability to engineer microorganisms for production of “designer” lipids and oils, wherein the fatty acid content and composition are carefully specified by genetic engineering, to produce oils having the specific oil content and composition that is sought for e.g., pharmaceuticals, dietary substitutes, medical foods, nutritional supplements, other food products, industrial oleochemicals and/or other end-use applications.

Most free fatty acids become esterified to coenzyme A [“CoA”], to yield acyl-CoAs. These molecules are then substrates for glycerolipid synthesis in the endoplasmic reticulum of the cell, where phosphatidic acid and diacylglycerol [“DAG”] are produced. Either of these metabolic intermediates may be directed to membrane phospholipids (e.g., phosphatidylglycerol, phosphatidylethanolamine, phosphatidylcholine) or DAG may be directed to form triacylglycerols [“TAGs”], the primary storage reserve of lipids in eukaryotic cells.

U.S. Pat. No. 7,267,976 describes the cloning of phospholipid:diacylglycerol acyltransferases [“PDAT”] and diacylglycerol acyltransferases (i.e., DGAT2) for altering PUFA and oil content in oleaginous yeast. U.S. Pat. No. 7,273,746 identifies nucleic acid fragments encoding diacylglycerol acyltransferases (i.e., DGAT1) and acyl-CoA:sterol-acyltransferases, useful for altering the quantity of oil in oleaginous microorganisms, such as oleaginous yeast.

Applicants' Assignee's copending published patent application U.S. 2006-0094088 teaches a method of manipulating DAG ATs and PDATs as a means to increase the percent of PUFAs, relative to the total fatty acids [“TFAs”], in the total lipid and oil fractions of an oleaginous organism.

Various mutant DGAT2 sequences derived from Yarrowia lipolytica are disclosed in Applicants' Assignee's International Application having Publication No. WO 2008/147935 which published on Dec. 4, 2008.

The disclosures cited above teach various enzymes and mechanisms that are useful for the recombinant production of PUFAs. However, commercial production of PUFAs will be enhanced by systems having increased rates of production, which may be effected by alterations in cellular carbon flux in the relevant pathways. There is a need to enhance the enzymatic activity of the relevant biosynthetic pathways to increase the overall rate of lipid and/or PUFA production.

It has been found that modifying expression of an acyl-CoA binding protein [“ACBP”] in oleaginous microbial organisms, can be used to regulate TFA content and/or fatty acid composition. Specifically, ACBP selectively binds medium and long chain acyl-CoA esters with high specificity and affinity. In vitro studies indicate that ACBP may regulate the availability of acyl-CoA esters in intermediary lipid metabolism. Various ACBP studies have been performed in yeast, such as Mandrup, S. et al. (Biochem. J., 290:369-374 (1993)), Knudsen, J. et al. (Biochem. J., 302(2):479-485 (1994)), Schjerling, C. K. et al. (J. Biol. Chem., 271:22514-22521 (1996)) and Gaigg, B. et al. (Mol. Biol. Cell, 12:1147-1160 (2001)). However, to date, no one has studied the effect of ACBP disruption or overexpression in an oleaginous organism, such as those engineered for high-level production of PUFAs.

### SUMMARY OF THE INVENTION

In one embodiment, the invention concerns an oleaginous microbial organism, comprising:
(i) a recombinant construct comprising at least one isolated polynucleotide comprising a nucleic acid sequence encoding an acyl-CoA binding protein operably linked to at least one regulatory sequence; and
(ii) a source of fatty acids.

The oleaginous microbial organism may additionally comprise a recombinant construct having at least one sequence encoding a diacylglycerol acyltransferase selected from the group consisting of DGAT1, DGAT2 and DGAT1 in combination with DGAT2.

In a second embodiment, the invention concerns an oleaginous microbial organism, comprising:
(i) a disruption in the native gene encoding an acyl-CoA binding protein; and
(ii) a source of fatty acids.

Also in a third embodiment, the invention concerns a method for modifying total lipid content in an oleaginous microbial organism, comprising:
a) providing an oleaginous microbial organism, comprising:
   (i) a recombinant construct comprising at least one isolated polynucleotide comprising a nucleic acid sequence encoding an acyl-CoA binding protein operably linked to at least one regulatory sequence; and
   (ii) a source of fatty acids;
b) growing the cell of step (a) under conditions whereby transfer of the fatty acids to lipid fractions of the organism is altered by altering expression of the isolated polynucleotide comprising a nucleic acid sequence encoding an acyl-CoA binding protein; and
c) optionally recovering the total lipid fractions of step (b).

In a fourth embodiment, the invention concerns a method for modifying fatty acid composition in an oleaginous microbial organism, comprising:
a) providing an oleaginous microbial organism, comprising:
   (i) a recombinant construct comprising at least one isolated polynucleotide comprising a nucleic acid sequence encoding an acyl-CoA binding protein operably linked to at least one regulatory sequence; and
   (ii) a functional ω-3/ω-6 fatty acid biosynthetic pathway comprising at least one isolated polynucleotide comprising a nucleic acid sequence encoding a desaturase enzyme for biosynthesis of fatty acids;
b) growing the cell of step (a) under conditions whereby the expression of the acyl-CoA binding protein is altered, resulting in an altered rate of desaturation by the at least one desaturase enzyme and an altered transfer of the synthesized fatty acids to lipid fractions of the organism;
c) optionally recovering the lipid fractions of step (b); and,
d) optionally determining the modified fatty acid composition of the lipid fractions of step (c).

In a fifth embodiment, the invention concerns a method for increasing the total lipid content in a Yarrowia sp., comprising:
a) providing Yarrowia sp., comprising:
   i) a recombinant construct comprising at least one isolated polynucleotide comprising a nucleic acid sequence encoding an acyl-CoA binding protein, said protein selected from the group consisting of:
      (a) a protein consisting essentially of the sequence set forth in SEQ ID NO:2; and
      (b) a protein comprising the following amino acid sequence motifs: SEQ ID NO:36, SEQ ID NO:37 and SEQ ID NO:38;
   wherein said isolated polynucleotide is operably linked to at least one regulatory sequence; and
   ii) a source of fatty acids;
b) growing the Yarrowia sp. of step (a) under conditions whereby the expression of the acyl-CoA binding protein results in an increased total lipid content of at least 5% when compared to the total lipid content of a Yarrowia sp. lacking said recombinant construct; and,
c) optionally recovering the total lipids of step (b).

### Biological Deposits

The following biological material has been deposited with the American Type Culture Collection (ATCC), 10801 University Boulevard, Manassas, Va. 20110-2209, and bears the following designation, accession number and date of deposit.

| Biological Material | Accession No. | Date of Deposit |
|---|---|---|
| Yarrowia lipolytica Y4128 | ATCC PTA-8614 | Aug. 23, 2007 |

The biological material listed above was deposited under the terms of the Budapest Treaty on the International Recognition of the Deposit of Microorganisms for the Purposes of Patent Procedure. The listed deposit will be maintained in the indicated international depository for at least 30 years and will be made available to the public upon the grant of a patent disclosing it. The availability of a deposit does not constitute a license to practice the subject invention in derogation of patent rights granted by government action.

### BRIEF DESCRIPTION OF THE DRAWINGS AND SEQUENCE LISTINGS

FIG. 1 is a representative ω-3 and ω-6 fatty acid biosynthetic pathway, and should be viewed together when considering the description of this pathway below.

FIG. 2 diagrams the development of Yarrowia lipolytica strain Y4305U.

FIG. 3 provides a plasmid map for pYPS161-ACBP.

FIG. 4 provides plasmid maps for the following: (A) pZP2-Pex; and, (B) pZP2-YACBP.

The invention can be more fully understood from the following detailed description and the accompanying sequence descriptions, which form a part of this application.

The following sequences comply with 37 C.F.R. §1.821-1.825 (“Requirements for Patent Applications Containing Nucleotide Sequences and/or Amino Acid Sequence Disclosures—the Sequence Rules”) and are consistent with World Intellectual Property Organization (WIPO) Standard ST.25 (1998) and the sequence listing requirements of the EPO and PCT (Rules 5.2 and 49.5(a-bis), and Section 208 and Annex C of the Administrative Instructions). The symbols and format used for nucleotide and amino acid sequence data comply with the rules set forth in 37 C.F.R. §1.822.

SEQ ID NOs:1-38 are ORFs encoding genes or proteins (or portions thereof), primers, or plasmids, as identified in Table 1.

TABLE 1
Summary Of Nucleic Acid And Protein SEQ ID Numbers

| | Nucleic acid | Protein |
|---|---|---|
| Description and Abbreviation | SEQ ID NO. | SEQ ID NO. |
| Yarrowia lipolytica ACBP | 1 (261 bp) | 2 (86 AA) |
| Yarrowia lipolytica DGAT1 | 3 (1581 bp) | 4 (526 AA) |
| Yarrowia lipolytica DGAT2 | 5 (2119 bp) | 6 (514 AA) |
| Yarrowia lipolytica DGAT2 | 7 (1545 bp) | 8 (514 AA) |
| Yarrowia lipolytica DGAT2 comprising | — | 9 (514 aa) |
| codon 326 mutated from Tyr to Phe | | |
| Yarrowia lipolytica DGAT2 comprising | — | 10 (514 aa) |
| codon 326 mutated from Tyr to Leu | | |
| Yarrowia lipolytica DGAT2 comprising | — | 11 (514 aa) |
| codon 327 mutated from Arg to Lys | | |
| Plasmid pYPS161 | 12 (7966 bp) | — |
| Plasmid pYPS161-ACBP | 13 (7334 bp) | — |
| PCR primer ACBPFii | 14 | — |
| PCR primer ACBPRii | 15 | — |
| PCR primer 3UTR-URA3 | 16 | — |
| PCR primer 3R-ACBPn | 17 | — |
| Real time PCR primer ef-324F | 18 | — |
| Real time PCR primer ef-392R | 19 | — |
| Real time PCR primer ACB1-378F | 20 | — |
| Real time PCR primer ACB1-474R | 21 | — |
| Nucleotide portion of TaqMan probe ef-345T | 22 | — |
| Nucleotide portion of TaqMan probe ACB1- | 23 | — |
| 398T | | |
| Primer ACBP-F | 24 | — |
| Primer ACBP-R | 25 | — |
| Plasmid pZP2-Pex10 | 26 (8784 bp) | — |
| Plasmid pZP2-YACBP | 27 (7899 bp) | — |
| Primer YDGAT1-F | 28 | — |
| Primer YDGAT1-R | 29 | — |
| Primer YDGAT2-F | 30 | — |
| Primer YDGAT2-R | 31 | — |
| Plasmid pFBAIN-MOD-1 | 32 (6991 bp) | — |
| Plasmid pFBAIN-YDGAT1 | 33 (8568 bp) | — |
| Plasmid pFBAIN-YDGAT2 | 34 (8532 bp) | — |
| Plasmid pZKUM | 35 (4313 bp) | — |
| Motif #1 | — | 36 |
| Motif #2 | — | 37 |
| Motif #3 | — | 38 |

### DETAILED DESCRIPTION OF THE INVENTION

New methods utilizing acyl-CoA binding protein [“ACBP”] enzymes and genes encoding the same are disclosed herein, that may be used for the manipulation of the lipid and oil content in oleaginous microbial organisms, particularly those oleaginous organisms producing polyunsaturated fatty acids [“PUFAs”] within their lipid and oil fractions.

PUFAs, or derivatives thereof, are used as dietary substitutes, or supplements, particularly infant formulas, for patients undergoing intravenous feeding or for preventing or treating malnutrition. Alternatively, the purified PUFAs (or derivatives thereof) may be incorporated into cooking oils, fats or margarines formulated so that in normal use the recipient would receive the desired amount for dietary supplementation. The PUFAs may also be incorporated into infant formulas, nutritional supplements or other food products and may find use as anti-inflammatory or cholesterol lowering agents. Optionally, the compositions may be used for pharmaceutical use (human or veterinary).

All patents, patent applications, and publications cited herein are incorporated by reference in their entirety.

In the context of this disclosure, a number of terms and abbreviations are used. The following definitions are provided.

“Co-enzyme A” is abbreviated CoA.

“Acyl-CoA binding protein” is abbreviated ACBP.

The term “invention” or “present invention” as used herein is not meant to be limiting to any one specific embodiment of the invention but applies generally to any and all embodiments of the invention as described in the claims and specification.

The term “fatty acids” refers to long-chain aliphatic acids (alkanoic acids) of varying chain lengths, from about C₁₂ to C₂₂, although both longer and shorter chain-length acids are known. The structure of a fatty acid is represented by a simple notation system of “X:Y”, where X is the total number of carbon [“C”] atoms in the particular fatty acid and Y is the number of double bonds. Additional details concerning the differentiation between “saturated fatty acids” versus “unsaturated fatty acids”, “monounsaturated fatty acids” versus “polyunsaturated fatty acids” [“PUFAs”], and “omega-6 fatty acids” [“ω-6” or “n-6”] versus “omega-3 fatty acids” [“ω-3” or “n-3”] are provided in U.S. Pat. No. 7,238,482, which is hereby incorporated herein by reference.

Nomenclature used to describe PUFAs herein is shown in Table 2. In the column titled “Shorthand Notation”, the omega-reference system is used to indicate the number of carbons, the number of double bonds and the position of the double bond closest to the omega carbon, counting from the omega carbon (which is numbered 1 for this purpose). The remainder of the Table summarizes the common names of ω-3 and ω-6 fatty acids and their precursors, the abbreviations that will be used throughout the specification and the chemical name of each compound.

TABLE 2
Nomenclature Of Polyunsaturated Fatty Acids And Precursors

| | | | Shorthand |
|---|---|---|---|
| Common Name | Abbreviation | Chemical Name | Notation |
| Myristic | — | tetradecanoic | 14:0 |
| Palmitic | Palmitate | hexadecanoic | 16:0 |
| Palmitoleic | — | 9-hexadecenoic | 16:1 |
| Stearic | — | octadecanoic | 18:0 |
| Oleic | — | cis-9-octadecenoic | 18:1 |
| Linoleic | LA | cis-9,12-octadecadienoic | 18:2 ω-6 |
| γ-Linolenic | GLA | cis-6,9,12-octadecatrienoic | 18:3 ω-6 |
| Eicosadienoic | EDA | cis-11,14-eicosadienoic | 20:2 ω-6 |
| Dihomo-γ- | DGLA | cis-8,11,14-eicosatrienoic | 20:3 ω-6 |
| linolenic | | | |
| Sciadonic | SCI | cis-5,11,14-eicosatrienoic | 20:3b ω-6 |
| Arachidonic | ARA | cis-5,8,11,14- | 20:4 ω-6 |
| | | eicosatetraenoic | |
| α-Linolenic | ALA | cis-9,12,15-octadecatrienoic | 18:3 ω-3 |
| Stearidonic | STA | cis-6,9,12,15- | 18:4 ω-3 |
| | | octadecatetraenoic | |
| Eicosatrienoic | ETrA | cis-11,14,17-eicosatrienoic | 20:3 ω-3 |
| Eicosa- | ETA | cis-8,11,14,17- | 20:4 ω-3 |
| tetraenoic | | eicosatetraenoic | |
| Juniperonic | JUP | cis-5,11,14,17- | 20:4b ω-3 |
| | | eicosatetraenoic | |
| Eicosa- | EPA | cis-5,8,11,14,17- | 20:5 ω-3 |
| pentaenoic | | eicosapentaenoic | |
| Docosatrienoic | DRA | cis-10,13,16-docosatrienoic | 22:3 ω-6 |
| Docosa- | DTA | cis-7,10,13,16- | 22:4 ω-6 |
| tetraenoic | | docosatetraenoic | |
| Docosa- | DPAn-6 | cis-4,7,10,13,16- | 22:5 ω-6 |
| pentaenoic | | docosapentaenoic | |
| Docosa- | DPA | cis-7,10,13,16,19- | 22:5 ω-3 |
| pentaenoic | | docosapentaenoic | |
| Docosa- | DHA | cis-4,7,10,13,16,19- | 22:6 ω-3 |
| hexaenoic | | docosahexaenoic | |

The term “oleaginous” refers to those organisms that tend to store their energy source in the form of lipid (or “oil”) (Weete, In: Fungal Lipid Biochemistry, 2^{nd} Ed., Plenum, 1980). And, for the purposes herein, oleaginous organisms include bacteria, algae, moss, yeast, fungi and plants that have the ability to produce oils.

The term “oleaginous yeast” refers to those microorganisms classified as yeasts that can make oils. Generally, the cellular oil or triacylglycerol content of oleaginous microorganisms follows a sigmoid curve, wherein the concentration of lipid increases until it reaches a maximum at the late logarithmic or early stationary growth phase and then gradually decreases during the late stationary and death phases (Yongmanitchai and Ward, Appl. Environ. Microbiol. 57:419-25 (1991)). It is not uncommon for oleaginous microorganisms to accumulate in excess of about 25% of their dry cell weight as oil. Examples of oleaginous yeast include, but are no means limited to, the following genera: Yarrowia, Candida, Rhodotorula, Rhodosporidium, Cryptococcus, Trichosporon and Lipomyces.

The term “total lipid fraction” of cells herein refers to all esterified fatty acids of the cell. Various subfractions within the total lipid fraction can be isolated, including the diacylglycerol, monoacylglycerol and triacylglycerol [“TAG” or “oil”] fractions, phosphatidylcholine fraction and the phosphatidyletanolamine fraction, although this is by no means inclusive of all sub-fractions.

The terms “triacylglycerols” [“TAGs”] and “oil” are interchangeable and refer to neutral lipids composed of three fatty acyl residues esterified to a glycerol molecule. TAGs can contain long chain PUFAs, as well as shorter saturated and unsaturated fatty acids and longer chain saturated fatty acids. The TAG fraction of cells is also referred to as the “oil fraction”, and “oil biosynthesis” generically refers to the synthesis of TAGs in the cell. The oil or TAG fraction is a sub-fraction of the total lipid fraction, although it also constitutes a major part of the total lipid content, measured as the weight of total fatty acids in the cell as a percent of the dry cell weight [infra], in oleaginous organisms. The fatty acid composition in the TAG fraction and the fatty acid composition of the total lipid fraction are generally similar. Thus, an increase or decrease in the concentration of PUFAs in the total lipid fraction will correspond with an increase or decrease in the concentration of PUFAs in the TAG fraction, and vice versa.

The term “total fatty acids” [“TFAs”] herein refers to the sum of all cellular fatty acids that can be derivitized to fatty acid methyl esters [“FAMEs”] by the base transesterification method (as known in the art) in a given sample, which may be the total lipid fraction or the oil fraction, for example. Thus, total fatty acids include fatty acids from neutral and polar lipid fractions, including the phosphatidylcholine fraction, the phosphatidyletanolamine fraction and the diacylglycerol, monoacylglycerol and triacylglycerol [“TAG” or “oil”] fractions but not free fatty acids.

The term “total lipid content” of cells is a measure of TFAs as a percent of the dry cell weight [“DCW”]. Thus, total lipid content [“TFAs % DCW”] is equivalent to, e.g., milligrams of total fatty acids per 100 milligrams of DCW.

Generally, the concentration of a fatty acid is expressed herein as a weight percent of TFAs [“% TFAs”], e.g., milligrams of the given fatty acid per 100 milligrams of TFAs. Unless otherwise specifically stated in the disclosure herein, reference to the percent of a given fatty acid with respect to total lipids is equivalent to concentration of the fatty acid as % TFAs (e.g., % EPA of total lipids is equivalent to EPA % TFAs).

In some cases, it is useful to express the content of a given fatty acid(s) in a cell as its percent of the dry cell weight [“% DCW”]. Thus, for example, eicosapentaenoic acid % DCW would be determined according to the following formula: (eicosapentaenoic acid % TFAs)*(TFA % DCW)]/100.

The terms “lipid profile” and “lipid composition” are interchangeable and refer to the amount of an individual fatty acid contained in a particular lipid fraction, such as in the total lipid fraction or the oil [“TAG”] fraction, wherein the amount is expressed as a percent of TFAs. The sum of each individual fatty acid present in the mixture should be 100.

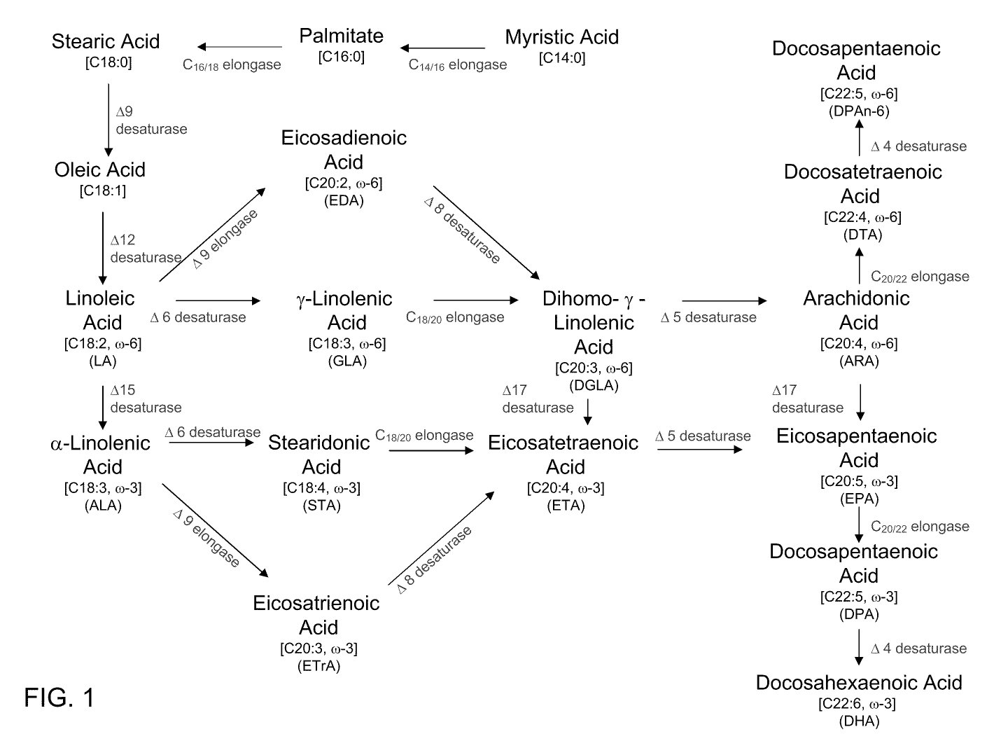

The term “acyl CoA binding protein” [“ACBP”] refers to a small (−10 kD) intracellular lipid-binding protein that is structurally and functionally conserved from yeast to mammals. ACBP selectively binds medium and long chain acyl-CoA esters with high specificity and affinity (although ACBP is unable to bind free fatty acids) and efficiently protects acyl-CoA esters from hydrolysis by thioesterases. In vitro studies indicate that ACBP may regulate the availability of acyl-CoA esters for intermediary lipid metabolism. Based on the high degree of sequence conservation, a significant number of ACBP proteins have been identified (see, e.g., FIG. 1 of PCT Publication No. WO 2002/061096 A1; Burton, M. et al., Biochem. J., 392:299-307 (2005)), which can generally be divided into 4 distinct groups: the generally expressed ACBP isoform, first isolated from bovine liver; the testis specific isoform (also called endozopine-like protein); a brain specific isoform; and, longer, membrane bound isoforms.

The term “YL ACBP” refers to the Yarrowia lipolytica gene encoding an acyl CoA binding protein (ORF YALI0E23185g within the public Y. lipolytica protein database of the “Yeast project Genolevures” (Center for Bioinformatics, LaBR1, Talence Cedex, France; see also Dujon, B. et al., Nature, 430(6995):35-44 (2004)). The nucleotide sequence of YL ACBP is set forth as SEQ ID NO:1, while the YL ACBP protein is provided as SEQ ID NO:2 herein.

The term “DAG AT” refers to a diacylglycerol acyltransferase (also known as an acyl-CoA-diacylglycerol acyltransferase or a diacylglycerol O-acyltransferase) (EC 2.3.1.20). This enzyme is responsible for the conversion of acyl-CoA and 1,2-diacylglycerol to TAG and CoA, thereby involved in the terminal step of TAG biosynthesis. Two families of DAG AT enzymes exist: DGAT1 and DGAT2. The former family shares homology with the acyl-CoA:cholesterol acyltransferase [“ACAT”] gene family, while the latter family is unrelated (Lardizabal et al., J. Biol. Chem. 276(42):38862-28869 (2001)).

A metabolic pathway, or biosynthetic pathway, in a biochemical sense, can be regarded as a series of chemical reactions occurring within a cell, catalyzed by enzymes, to achieve either the formation of a metabolic product to be used or stored by the cell, or the initiation of another metabolic pathway (then called a flux generating step). Many of these pathways are elaborate, and involve a step by step modification of the initial substance to shape it into a product having the exact chemical structure desired.

The term “PUFA biosynthetic pathway” refers to a metabolic process that converts oleic acid to ω-6 fatty acids such as LA, EDA, GLA, DGLA, ARA, DRA, DTA and DPAn-6 and ω-3 fatty acids such as ALA, STA, ETrA, ETA, EPA, DPA and DHA. This process is well described in the literature (e.g., see U.S. Pat. Pub. No. 2006-0115881-A1). Briefly, this process involves elongation of the carbon chain through the addition of carbon atoms and desaturation of the molecule through the addition of double bonds, via a series of special desaturation and elongation enzymes termed “PUFA biosynthetic pathway enzymes” that are present in the endoplasmic reticulim membrane. More specifically, “PUFA biosynthetic pathway enzyme” refers to any of the following enzymes (and genes which encode said enzymes) associated with the biosynthesis of a PUFA, including: Δ9 elongase, C_{14/16} elongase, C_{16/18} elongase, C_{18/20} elongase, C_{20/22} elongase, Δ4 desaturase, Δ5 desaturase, Δ6 desaturase, Δ12 desaturase, Δ15 desaturase, Δ17 desaturase, Δ9 desaturase and/or Δ8 desaturase.

The term “functional” as used herein relating to the PUFA biosynthetic pathway, for synthesis of ω-3/ω-6 fatty acids, means that some (or all) of the genes in the pathway express active enzymes, resulting in in vivo catalysis or substrate conversion. It should be understood that “ω-3/ω-6 fatty acid biosynthetic pathway” or “functional ω-3/ω-6 fatty acid biosynthetic pathway” does not imply that all of the PUFA biosynthetic pathway genes in the above paragraph are required, as a number of fatty acid products will require only the expression of a subset of the genes of this pathway.

The term “desaturase” refers to a polypeptide that can desaturate, i.e., introduce a double bond, in one or more fatty acids to produce a fatty acid or precursor of interest. Despite use of the omega-reference system throughout the specification to refer to specific fatty acids, it is more convenient to indicate the activity of a desaturase by counting from the carboxyl end of the substrate using the delta-system. Of particular interest herein are Δ6 desaturases, Δ8 desaturases, Δ5 desaturases, Δ4 desaturases, Δ12 desaturases, Δ15 desaturases, Δ17 desaturases and Δ9 desaturases. In the art, Δ15 and Δ17 desaturases are also occasionally referred to as “omega-3 desaturases”, “ω-3 desaturases” and/or “ω-3 desaturases”, based on their ability to convert ω-6 fatty acids into their ω-3 counterparts.

The term “elongase” refers to a polypeptide that can elongate a fatty acid carbon chain to produce an acid that is 2 carbons longer than the fatty acid substrate that the elongase acts upon. This process of elongation occurs in a multi-step mechanism in association with fatty acid synthase, as described in U.S. Patent Publication No. 2005/0132442. Examples of reactions catalyzed by elongase systems are the conversion of GLA to DGLA, STA to ETA, LA to EDA, ALA to ETrA, ARA to DTA and EPA to DPA. In general, the substrate selectivity of elongases is somewhat broad but segregated by both chain length and the degree of unsaturation. For example, a C_{14/16} elongase will utilize a C₁₄ substrate (e.g., myristic acid), a C_{16/18} elongase will utilize a C₁₆ substrate (e.g., palmitate), a C_{18/20} elongase (also known as a Δ6 elongase as the terms can be used interchangeably) will utilize a C₁₈ substrate (e.g., GLA, STA) and a C_{20/22} elongase will utilize a C₂₀ substrate (e.g., ARA, EPA). In like manner, a Δ9 elongase catalyzes the conversion of LA to EDA and/or ALA to ETrA.

The terms “conversion efficiency” and “percent substrate conversion” refer to the efficiency by which a particular enzyme (e.g., a desaturase) can convert substrate to product. The conversion efficiency is measured according to the following formula: ([product]/[substrate+product])*100, where ‘product’ includes the immediate product and all products in the pathway derived from it.

The term “disruption”, in or in connection with a native ACBP, refers to down-regulation, either partial or total, of a gene encoding ACBP which can result in altering the activity of ACBP. For example, disruption includes, but is not limited to, an insertion, deletion, or targeted mutation within a portion of that gene, that results in either a complete gene knockout such that the gene is deleted from the genome and no protein is translated or a translated ACBP having an insertion, deletion, amino acid substitution or other targeted mutation. The location of the disruption in the protein may be, for example, within the N-terminal portion of the protein or within the C-terminal portion of the protein. The disrupted ACBP will have altered activity with respect to the ACBP that was not disrupted. The alteration in activity can be a decrease in activity whereby the ACBP has some level of activity up to and including losing activity altogether, i.e., the ACBP has no detectable level of activity (it appears to be non-functional. A disruption in a native gene encoding an ACBP also includes alternate means that result in low or lack of expression of the ACBP, such as could result via manipulating the regulatory sequences, transcription and translation factors and/or signal transduction pathways or by use of sense, antisense or RNAi technology, etc.

The terms “polynucleotide”, “polynucleotide sequence”, “nucleic acid sequence”, “nucleic acid fragment” and “isolated nucleic acid fragment” are used interchangeably herein. These terms encompass nucleotide sequences and the like. A polynucleotide may be a polymer of RNA or DNA that is single- or double-stranded, that optionally contains synthetic, non-natural or altered nucleotide bases. A polynucleotide in the form of a polymer of DNA may be comprised of one or more segments of cDNA, genomic DNA, synthetic DNA, or mixtures thereof. Nucleotides (usually found in their 5′-monophosphate form) are referred to by a single letter designation as follows: “A” for adenylate or deoxyadenylate (for RNA or DNA, respectively), “C” for cytidylate or deoxycytidylate, “G” for guanylate or deoxyguanylate, “U” for uridylate, “T” for deoxythymidylate, “R” for purines (A or G), “Y” for pyrimidines (C or T), “K” for G or T, “H” for A or C or T, “I” for inosine, and “N” for any nucleotide.

A “substantial portion” of an amino acid or nucleotide sequence is that portion comprising enough of the amino acid sequence of a polypeptide or the nucleotide sequence of a gene to putatively identify that polypeptide or gene, either by manual evaluation of the sequence by one skilled in the art, or by computer-automated sequence comparison and identification using algorithms such as BLAST (Basic Local Alignment Search Tool; Altschul, S. F., et al., J. Mol. Biol., 215:403-410 (1993)). In general, a sequence of ten or more contiguous amino acids or thirty or more nucleotides is necessary in order to identify putatively a polypeptide or nucleic acid sequence as homologous to a known protein or gene. Moreover, with respect to nucleotide sequences, gene-specific oligonucleotide probes comprising 20-30 contiguous nucleotides may be used in sequence-dependent methods of gene identification (e.g., Southern hybridization) and isolation, such as in situ hybridization of bacterial colonies or bacteriophage plaques. In addition, short oligonucleotides of 12-15 bases may be used as amplification primers in PCR in order to obtain a particular nucleic acid fragment comprising the primers. Accordingly, a “substantial portion” of a nucleotide sequence comprises enough of the sequence to specifically identify and/or isolate a nucleic acid fragment comprising the sequence.

The term “conserved domain” or “motif” means a set of amino acids conserved at specific positions along an aligned sequence of evolutionarily related proteins. While amino acids at other positions can vary between homologous proteins, amino acids that are highly conserved at specific positions indicate amino acids that are essential in the structure, the stability, or the activity of a protein. Because they are identified by their high degree of conservation in aligned sequences of a family of protein homologues, they can be used as identifiers, or “signatures”, to determine if a protein with a newly determined sequence belongs to a previously identified protein family. Motifs that are found in most ACBPs include Leu-Xaa-Xaa-Tyr-Xaa-Xaa-(Tyr/Phe)-Lys (LxxYxx[Y/F]K; SEQ ID NO:36), Lys-Xaa-Xaa-Ala-Trp (KxxAW; SEQ ID NO:37) and Ala-Xaa-Xaa-Xaa-Tyr (AxxxY; SEQ ID NO:38).

The terms “homology”, “homologous”, “substantially similar” and “corresponding substantially” are used interchangeably herein. They refer to nucleic acid fragments wherein changes in one or more nucleotide bases do not affect the ability of the nucleic acid fragment to mediate gene expression or produce a certain phenotype. These terms also refer to modifications of the nucleic acid fragments such as deletion or insertion of one or more nucleotides that do not substantially alter the functional properties of the resulting nucleic acid fragment relative to the initial, unmodified fragment.

Moreover, the skilled artisan recognizes that substantially similar nucleic acid sequences are also defined by their ability to hybridize (under moderately stringent conditions, e.g., 0.5×SSC, 0.1% SDS, 60° C.) with the ACBP sequences described herein, or to any portion of the nucleotide sequences disclosed herein and which are functionally equivalent to any of the nucleic acid sequences disclosed herein. Stringency conditions can be adjusted to screen for moderately similar fragments, such as homologous sequences from distantly related organisms, to highly similar fragments, such as genes that duplicate functional enzymes from closely related organisms. Post-hybridization washes determine stringency conditions. An extensive guide to the hybridization of nucleic acids is found in Tijssen, Laboratory Techniques in Biochemistry and Molecular Biology—Hybridization with Nucleic Acid Probes, Part I, Chapter 2 “Overview of principles of hybridization and the strategy of nucleic acid probe assays”, Elsevier, New York (1993); and Current Protocols in Molecular Biology, Chapter 2, Ausubel et al., Eds., Greene Publishing and Wiley-Interscience, New York (1995).

As used herein, the term “percent identity” refers to a relationship between two or more polypeptide sequences or two or more polynucleotide sequences, as determined by comparing the sequences. “Identity” also means the degree of sequence relatedness between polypeptide or polynucleotide sequences, as the case may be, as determined by the percentage of match between compared sequences. “Percent identity” and “percent similarity” can be readily calculated by known methods, including but not limited to those described in: 1) Computational Molecular Biology (Lesk, A. M., Ed.) Oxford University: NY (1988); 2) Biocomputing: Informatics and Genome Projects (Smith, D. W., Ed.) Academic: NY (1993); 3) Computer Analysis of Sequence Data, Part I (Griffin, A. M., and Griffin, H. G., Eds.) Humania: NJ (1994); 4) Sequence Analysis in Molecular Biology (von Heinje, G., Ed.) Academic (1987); and, 5) Sequence Analysis Primer (Gribskov, M. and Devereux, J., Eds.) Stockton: NY (1991).

Preferred methods to determine percent identity are designed to give the best match between the sequences tested. Methods to determine percent identity and percent similarity are codified in publicly available computer programs. Sequence alignments and percent identity calculations may be performed using the MegAlign™ program of the LASERGENE bioinformatics computing suite (DNASTAR Inc., Madison, Wis.). Multiple alignment of the sequences is performed using the “Clustal method of alignment” which encompasses several varieties of the algorithm including the “Clustal V method of alignment” and the “Clustal W method of alignment” (described by Higgins and Sharp, CABIOS, 5:151-153 (1989); Higgins, D. G. et al., Comput. Appl. Biosci., 8:189-191 (1992)) and found in the MegAlign™ v6.1 program of the LASERGENE bioinformatics computing suite (DNASTAR Inc.). After alignment of the sequences using either Clustal program, it is possible to obtain a “percent identity” by viewing the “sequence distances” table in the program.

It is well understood by one skilled in the art that many levels of sequence identity are useful in identifying polypeptides, from other species, wherein such polypeptides have the same or similar function or activity. Useful examples of percent identities include any integer percentage from 50% to 100%, such as 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99%. Also, of interest is any full-length or partial complement of this isolated nucleotide fragment.

“Codon degeneracy” refers to the nature in the genetic code permitting variation of the nucleotide sequence without effecting the amino acid sequence of an encoded polypeptide. The skilled artisan is well aware of the “codon-bias” exhibited by a specific host cell in usage of nucleotide codons to specify a given amino acid. Therefore, when synthesizing a gene for improved expression in a host cell, it is desirable to design the gene such that its frequency of codon usage approaches the frequency of preferred codon usage of the host cell.

“Synthetic genes” can be assembled from oligonucleotide building blocks that are chemically synthesized using procedures known to those skilled in the art. These building blocks are ligated and annealed to form gene segments that are then enzymatically assembled to construct the entire gene. Accordingly, the genes can be tailored for optimal gene expression based on optimization of nucleotide sequence to reflect the codon bias of the host cell. The skilled artisan appreciates the likelihood of successful gene expression if codon usage is biased towards those codons favored by the host. Determination of preferred codons can be based on a survey of genes derived from the host cell, where sequence information is available.

“Gene” refers to a nucleic acid fragment that expresses a specific protein, and that may refer to the coding region alone or may include regulatory sequences preceding (5′ non-coding sequences) and following (3′ non-coding sequences) the coding sequence. “Native gene” refers to a gene as found in nature with its own regulatory sequences. “Chimeric gene” refers to any gene that is not a native gene, comprising regulatory and coding sequences that are not found together in nature. Accordingly, a chimeric gene may comprise regulatory sequences and coding sequences that are derived from different sources, or regulatory sequences and coding sequences derived from the same source, but arranged in a manner different than that found in nature. “Endogenous gene” refers to a native gene in its natural location in the genome of an organism. A “foreign” gene refers to a gene not normally found in the host organism, but that is introduced into the host organism by gene transfer. Foreign genes can comprise native genes inserted into a non-native organism, or chimeric genes. A “transgene” is a gene that has been introduced into the genome by a transformation procedure. A “codon-optimized gene” is a gene having its frequency of codon usage designed to mimic the frequency of preferred codon usage of the host cell.

“Coding sequence” refers to a DNA sequence that codes for a specific amino acid sequence. “Regulatory sequences” refer to nucleotide sequences located upstream (5′ non-coding sequences), within, or downstream (3′ non-coding sequences) of a coding sequence, and which influence the transcription, RNA processing or stability, or translation of the associated coding sequence. Regulatory sequences may include promoters, enhancers, silencers; 5′ untranslated leader sequence (e.g., between the transcription start site and the translation initiation codon), introns, polyadenylation recognition sequences, RNA processing sites, effector binding sites and stem-loop structures.

“Promoter” refers to a DNA sequence capable of controlling the expression of a coding sequence or functional RNA. In general, a coding sequence is located 3′ to a promoter sequence. Promoters may be derived in their entirety from a native gene, or be composed of different elements derived from different promoters found in nature, or even comprise synthetic DNA segments. It is understood by those skilled in the art that different promoters may direct the expression of a gene in different tissues or cell types, or at different stages of development, or in response to different environmental conditions. Promoters that cause a gene to be expressed in most cell types at most times are commonly referred to as “constitutive promoters”. It is further recognized that since in most cases the exact boundaries of regulatory sequences have not been completely defined, DNA fragments of different lengths may have identical promoter activity.

The terms “3′ non-coding sequence” and “transcription terminator” refer to DNA sequences located downstream of a coding sequence. This includes polyadenylation recognition sequences and other sequences encoding regulatory signals capable of affecting mRNA processing or gene expression. The polyadenylation signal is usually characterized by affecting the addition of polyadenylic acid tracts to the 3′ end of the mRNA precursor. The 3′ region can influence the transcription, RNA processing or stability, or translation of the associated coding sequence.

“RNA transcript” refers to the product resulting from RNA polymerase-catalyzed transcription of a DNA sequence. When the RNA transcript is a perfect complementary copy of the DNA sequence, it is referred to as the primary transcript. A RNA transcript is referred to as the mature RNA when it is a RNA sequence derived from post-transcriptional processing of the primary transcript. “Messenger RNA” or “mRNA” refers to the RNA that is without introns and that can be translated into protein by the cell. “cDNA” refers to a DNA that is complementary to, and synthesized from, a mRNA template using the enzyme reverse transcriptase. The cDNA can be single-stranded or converted into double-stranded form using the Klenow fragment of DNA polymerase I.

The term “operably linked” refers to the association of nucleic acid sequences on a single nucleic acid fragment so that the function of one is affected by the other. For example, a promoter is operably linked with a coding sequence when it is capable of affecting the expression of that coding sequence. That is, the coding sequence is under the transcriptional control of the promoter. Coding sequences can be operably linked to regulatory sequences in a sense or antisense orientation.

The term “recombinant” refers to an artificial combination of two otherwise separated segments of sequence, e.g., by chemical synthesis or by the manipulation of isolated segments of nucleic acids by genetic engineering techniques.

The term “expression”, as used herein, refers to the transcription and stable accumulation of sense (mRNA) or antisense RNA derived from the nucleic acid fragments. Expression may also refer to translation of mRNA into a protein (either precursor or mature).

The terms “plasmid” and “vector” refer to an extra chromosomal element often carrying genes that are not part of the central metabolism of the cell, and usually in the form of circular double-stranded DNA fragments. Such elements may be autonomously replicating sequences, genome integrating sequences, phage or nucleotide sequences, linear or circular, of a single- or double-stranded DNA or RNA, derived from any source, in which a number of nucleotide sequences have been joined or recombined into a unique construction which is capable of introducing an expression cassette(s) into a cell.

The term “expression cassette” refers to a fragment of DNA comprising the coding sequence of a selected gene and regulatory sequences preceding (5′ non-coding sequences) and following (3′ non-coding sequences) the coding sequence that are required for expression of the selected gene product. Thus, an expression cassette is typically composed of: 1) a promoter sequence; 2) a coding sequence (i.e., ORF); and, 3) a 3′ untranslated region (i.e., a terminator) that, in eukaryotes, usually contains a polyadenylation site. The expression cassette(s) is usually included within a vector, to facilitate cloning and transformation. Different expression cassettes can be transformed into different organisms including bacteria, yeast, plants and mammalian cells, as long as the correct regulatory sequences are used for each host.

“Transformation” refers to the transfer of a nucleic acid molecule into a host organism, resulting in genetically stable inheritance. The nucleic acid molecule may be a plasmid that replicates autonomously, for example, or, it may integrate into the genome of the host organism. Host organisms containing the transformed nucleic acid fragments are referred to as “transgenic” or “recombinant” or “transformed” organisms.

Standard recombinant DNA and molecular cloning techniques used herein are well known in the art and are described more fully in Sambrook, J., Fritsch, E. F. and Maniatis, T. Molecular Cloning: A Laboratory Manual; Cold Spring Harbor Laboratory: Cold Spring Harbor, N.Y. (1989); by Silhavy, T. J., Bennan, M. L. and Enquist, L. W., Experiments with Gene Fusions, Cold Spring Harbor Laboratory: Cold Spring Harbor, N.Y. (1984); and by Ausubel, F. M. et al., Current Protocols in Molecular Biology, published by Greene Publishing Assoc. and Wiley-Interscience, Hoboken, N.J. (1987). Transformation methods are well known to those skilled in the art and are described infra.

TAGs (the primary storage unit for fatty acids) are formed by a series of reactions that involve: 1) the esterification of one molecule of acyl-CoA to glycerol-3-phosphate via an acyltransferase to produce lysophosphatidic acid; 2) the esterification of a second molecule of acyl-CoA via an acyltransferase to yield 1,2-diacylglycerol phosphate (commonly identified as phosphatidic acid); 3) removal of a phosphate by phosphatidic acid phosphatase to yield 1,2-diacylglycerol [“DAG”]; and, 4) the addition of a third fatty acid by the action of a DAG AT (e.g., PDAT, DGAT1 or DGAT2) to form TAG.

A wide spectrum of fatty acids can be incorporated into TAGs, including saturated and unsaturated fatty acids and short-chain and long-chain fatty acids. Preferably, incorporation of “long-chain” PUFAs into TAG is most desirable, wherein long-chain PUFAs include any fatty acid derived from an 18:1 substrate having at least 18 carbons in length (i.e., C₁₈ or greater). This also includes hydroxylated fatty acids, epoxy fatty acids and conjugated linoleic acid.

Although most PUFAs are incorporated into TAGs as neutral lipids and are stored in lipid bodies, it is important to note that a measurement of the total lipids (or total fatty acid content) within an oleaginous organism should include those lipids that are located in the phosphatidylcholine [“PC”] fraction, phosphatidyletanolamine [“PE”] fraction, and triacylglycerol [“TAG”] fraction.

The metabolic process wherein oleic acid is converted to ω-3/ω-6 fatty acids involves elongation of the carbon chain through the addition of carbon atoms and desaturation of the molecule through the addition of double bonds. This requires a series of special desaturation and elongation enzymes present in the endoplasmic reticulim membrane. However, as seen in FIG. 1 and as described below, there are often multiple alternate pathways for production of a specific ω-3/ω-6 fatty acid.

These pathways start with the conversion of oleic acid to LA, the first of the ω-6 fatty acids, by a Δ12 desaturase. Then, using the “Δ6 desaturase/Δ6 elongase pathway” and linoleic acid [“LA”] as substrate, long chain ω-6 fatty acids are formed as follows: 1) LA is converted to γ-linolenic acid [“GLA”] by a Δ6 desaturase; 2) GLA is converted to dihomo-γ-linolenic acid [“DGLA”] by a C_{18/20} elongase; 3) DGLA is converted to arachidonic acid [“ARA”] by a A5 desaturase; 4) ARA is converted to docosatetraenoic acid [“DTA”] by a C_{20/22} elongase; and, 5) DTA is converted to docosapentaenoic acid [“DPAn-6”] by a Δ4 desaturase. Alternatively, the “Δ6 desaturase/Δ6 elongase” can use α-linolenic acid [“ALA”] as substrate to produce long chain ω-3 fatty acids as follows: 1) LA is converted to ALA, the first of the ω-3 fatty acids, by a Δ15 desaturase; 2) ALA is converted to stearidonic acid [“STA”] by a A6 desaturase; 3) STA is converted to eicosatetraenoic acid [“ETA”] by a C_{18/20} elongase; 4) ETA is converted to eicosapentaenoic acid [“EPA”] by a Δ5 desaturase; 5) EPA is converted to docosapentaenoic acid [“DPA”]by a C_{20/22} elongase; and, 6) DPA is converted to docosahexaenoic acid [“DHA”] by a Δ4 desaturase. Optionally, ω-6 fatty acids may be converted to ω-3 fatty acids; for example, ETA and EPA are produced from DGLA and ARA, respectively, by Δ17 desaturase activity.

Alternate pathways for the biosynthesis of ω-3/ω-6 fatty acids utilize a Δ9 elongase and Δ8 desaturase (i.e., the “Δ9 elongase/Δ8 desaturase pathway”). More specifically, LA and ALA may be converted to eicosadienoic acid [“EDA”] and eicosatrienoic acid [“ETrA”], respectively, by a Δ9 elongase; then, a Δ8 desaturase converts EDA to DGLA and/or ETrA to ETA. Downstream PUFAs are subsequently formed as described above.

It is contemplated that the particular functionalities required to be introduced into a specific host organism for production of ω-3/ω-6 fatty acids will depend on the host cell (and its native PUFA profile and/or desaturase/elongase profile), the availability of substrate, and the desired end product(s). For example, expression of the Δ6 desaturase/Δ6 elongase pathway may be preferred in some embodiments, as opposed to expression of the Δ9 elongase/Δ8 desaturase pathway, since PUFAs produced via the former pathway are not devoid of GLA and/or STA.

As previously defined, ACBP selectively binds medium and long chain acyl-CoA esters with high specificity and affinity and efficiently protects acyl-CoA esters from hydrolysis by thioesterases. In vitro studies indicate that ACBP may regulate the availability of acyl-CoA esters in intermediary lipid metabolism.

In yeast, ACBP is involved in fatty acid chain elongation, sphingolipid biosynthesis, protein sorting, and vesicular trafficking. More specifically, previous studies have determined that overexpression of ACBP in the yeast Saccharomyces cerevisiae significantly increases the acyl-CoA pool size, indicating that ACBP can generate an intracellular acyl-CoA pool (Mandrup, S., et al., Biochem. J., 290:369-374 (1993); Knudsen, J., et al., Biochem J., 302(2):479-485 (1994)). Similarly, disruption of ACBP in S. cerevisiae has been found to perturb acyl-CoA metabolism (Schjerling, C. K., et al., J. Biol. Chem., 271:22514-22521 (1996)) and modify composition of long-chain acyl-CoA (Gaigg, B., et al., Mol. Biol. Cell, 12:1147-1160 (2001))). Most recently, in the review by Schroeder, F., et al. (Lipids, 43:1-17 (2008)), it is noted that ACBP may also selectively cooperate with a nuclear receptor (i.e., HNF4α), to provide a signaling pathway for long-chain fatty acid metabolism. ACBP enhances the uptake of lipidic ligands (i.e., long-chain fatty acid CoAs), binds these ligands with high affinity in the cytoplasm, cotransports this cargo to nuclei and through the nuclear pores into the nucleoplasm, forms complexes with nuclear receptors exhibiting even higher affinity for the respective ligands, and directly channels this cargo to the respective nuclear receptors to regulate receptor activation. Thus, ACBP is suggested to act as nutrient sensor and in part regulate its own expression. To date, no one has studied the effect of ACBP disruption or overexpression in an oleaginous organism, such as those engineered for high-level production of PUFAs.

In one embodiment, the instant invention concerns an oleaginous microbial organism, comprising:
i) a recombinant construct comprising at least one isolated polynucleotide comprising a nucleic acid sequence encoding an acyl-CoA binding protein operably linked to at least one regulatory sequence; and
ii) a source of fatty acids.

In a second embodiment, the instant invention concerns an oleaginous microbial organism, comprising:
i) a disruption in a native gene encoding an acyl-CoA binding protein; and
ii) a source of fatty acids.

Sources of the oleaginous microbial organism can be selected from the group consisting of Yarrowia, Candida, Rhodotorula, Rhodosporidium, Cryptococcus, Trichosporon and Lipomyces.

In another aspect, the oleaginous microbial organism of the invention can comprise a recombinant construct having at least one nucleic acid sequence encoding a diacylglycerol acyltransferase selected from the group consisting of DGAT1, DGAT2 and DGAT1 in combination with DGAT2.

Numerous techniques are available to one of skill in the art for disrupting expression of an oleaginous microbial organism's native ACBP.

Generally the expression of a gene can be reduced or eliminated (i.e., “knocked out”) by, for example: 1) disrupting the gene expression through insertion, substitution and/or deletion of all or part of the target gene; 2) using antisense or iRNA technology; 3) using a host cell which naturally has [or has been mutated to have] little or none of the specific gene's activity; 4) over-expressing a mutagenized heterosubunit (i.e., in an enzyme that comprises two or more heterosubunits) to thereby reduce the enzyme's activity as a result of the “dominant negative effect”; and 5) manipulating the regulatory sequences controlling the expression of the protein.

Each of these techniques are briefly described in WO 2006/052912, the disclosure of which is hereby incorporated by reference.

In preferred embodiments, a foreign DNA fragment (typically a selectable marker gene) is inserted into the structural gene to be disrupted (i.e., ACBP) in order to interrupt its coding sequence and thereby functionally inactivate the gene. Transformation of the disruption cassette into the host cell results in replacement of the functional native gene by homologous recombination with the non-functional disrupted gene (see, for example: Hamilton et al., J. Bacteriol., 171:4617-4622 (1989); Balbas et al., Gene, 136:211-213 (1993); Gueldener et al., Nucleic Acids Res., 24:2519-2524 (1996); and Smith et al., Methods Mol. Cell. Biol., 5:270-277 (1996)). One skilled in the art will be familiar with the many techniques available for targeting a gene, thereby permitting positive or negative selection, creation of gene knockouts, and insertion of exogenous DNA sequences into specific genome sites in mammalian systems, plant cells, filamentous fungi, and/or microbial systems.

In alternate embodiments, the regulatory sequences associated with an ACBP coding sequence (e.g., promoters, translation leader sequences, introns, enhancers, initiation control regions, polyadenylation recognition sequences, RNA processing sites, effector binding sites and stem-loop structures) could be manipulated to result in diminished expression of the ACBP. For example, the ACBP promoter could be deleted or disrupted; or the native promoter driving expression of ACBP could be substituted with a heterologous promoter having diminished promoter activity with respect to the native promoter. Methods useful for manipulating regulatory sequences are well known to those skilled in the art.

An ACBP comprising the amino acid sequence set forth in SEQ ID NO:2 is disclosed herein, although variants and/or functional equivalents thereof are also envisioned to be suitable. Specifically, based on the substantial sequence conservation within ACBP proteins, it is well within the means of one of skill in the art to easily identify homologous proteins in other species and even novel proteins having essentially the same affinity for CoA esters of hydrophobic acids. All of these proteins and their functional variants are useful in the methods described below.

For example, Burton, M. et al. (Biochem. J., 392:299-307 (2005)) provides excellent sequence comparisons of ACBPs from vertebrates, urochordates, echinoderms, arthropods, nematodes and other lower metazoans, fungal and plant species, including e.g., GenBank Accession Nos., BI191959, BU065460, EM53049, EAA33144m BE776849, CD051645, CD044063, AAB31936, NC_{—}001139, AAC101000271, AABZ01000388, CAA69946, MBY01000137, Y08690, NP_{—}596820, CAE74488, CAE70798, CAE69296, NP_{—}491412, NP_{—}509822, NP_{—}498609 and NP_{—}496552. Based on the alignments therein, ACBP motifs are readily identified for use in the identification of homologous proteins in other species. A preferred set of motifs would include the following: Leu-Xaa-Xaa-Tyr-Xaa-Xaa-(Tyr/Phe)-Lys (LxxYxx[Y/F]K; SEQ ID NO:36), Lys-Xaa-Xaa-Ala-Trp (KxxAW; SEQ ID NO:37) and Ala-Xaa-Xaa-Xaa-Tyr (AxxxY; SEQ ID NO:38).

In another embodiment, a method for modifying total lipid content in an oleaginous microbial organism is provided herein, comprising:
a) providing an oleaginous microbial organism, comprising:
   (i) a recombinant construct comprising at least one isolated polynucleotide comprising a nucleic acid sequence encoding an acyl-CoA binding protein operably linked to at least one regulatory sequence; and
   (ii) a source of fatty acids;
b) growing the cell of step (a) under conditions whereby transfer of the fatty acids to lipid fractions of the organism is altered by altering expression of the isolated polynucleotide comprising a nucleic acid sequence encoding an acyl-CoA binding protein; and
c) optionally recovering the total lipid fractions of step (b).

This method permits expression of the at least one isolated polynucleotide comprising a nucleic acid sequence encoding an ACBP to either be decreased (thereby resulting in decreased total lipid content in the oleaginous microbial organism) or increased (thereby resulting in increased total lipid content in the oleaginous microbial organism).

Furthermore, a method for modifying fatty acid composition in an oleaginous microbial organism is provided, comprising:
(a) providing an oleaginous microbial organism, comprising:
(i) a recombinant construct comprising at least one isolated polynucleotide comprising a nucleic acid sequence encoding an acyl-CoA binding protein operably linked to at least one regulatory sequence; and
(ii) a functional ω-3/ω-6 fatty acid biosynthetic pathway comprising at least one isolated polynucleotide comprising a nucleic acid sequence encoding a desaturase enzyme for biosynthesis of fatty acids;
(b) growing the cell of step (a) under conditions whereby the expression of the acyl-CoA binding protein is altered, resulting in an altered rate of desaturation by the at least one desaturase enzyme and an altered transfer of the synthesized fatty acids to lipid fractions of the organism;
(c) optionally recovering the lipid fractions of step (b); and,
(d) optionally determining the modified fatty acid composition of the lipid fractions of step (c).

Again, this method permits the expression of the at least one isolated polynucleotide comprising a nucleic acid sequence encoding an ACBP to either be decreased or increased. When the expression is decreased, there is an increased rate of desaturation, thereby causing increased production of PUFAs as a percent of the total fatty acids, and a decrease in total lipid content in the oleaginous microbial organism. In contrast, when the expression of ACBP is increased, a decreased rate of desaturation results, thereby causing decreased production of PUFAs as a percent of the total fatty acids, and an increase in total lipid content in the oleaginous microbial organism.

It is believed that by down-regulating activity of ACBP, the substrate competition between oil biosynthesis and polyunsaturation is reduced in favor of polyunsaturation during oleaginy; thus, in effect, when the activity of ACBP is diminished or knocked out, polyunsaturation is permitted to occur more efficiently.

Furthermore, the results achieved by manipulation of ACBP may be enhanced when a DAG AT (i.e., DGAT1, DGAT2) enzyme is similarly manipulated.

Microbial expression systems and expression vectors containing regulatory sequences that direct high level expression of foreign proteins (i.e., ACBP) are well known to those skilled in the art. Any of these could be used to construct chimeric ACBP genes that could then be introduced into appropriate oleaginous microorganisms via transformation to provide high-level expression of the encoded ACBP enzyme.

Vectors (e.g., constructs, plasmids) and DNA expression cassettes useful for the transformation of suitable microbial host cells are well known in the art. The specific choice of sequences present in the construct is dependent upon the desired expression products (supra), the nature of the host cell and the proposed means of separating transformed cells versus non-transformed cells. Typically, however, the vector contains at least one expression cassette, a selectable marker and sequences allowing autonomous replication or chromosomal integration. Suitable expression cassettes comprise a region 5′ of the gene that controls transcription (e.g., a promoter), the gene coding sequence, and a region 3′ of the DNA fragment that controls transcriptional termination (i.e., a terminator). It is most preferred when both control regions are derived from genes from the transformed microbial host cell, although it is to be understood that such control regions need not be derived from the genes native to the specific species chosen as a production host.

Transcriptional control regions (also initiation control regions or promoters) which are useful to drive expression of the ACBP ORFs in the desired microbial host cell are numerous and familiar to those skilled in the art. Virtually any promoter (i.e., native, synthetic, or chimeric) capable of directing expression of these genes in the selected host cell is suitable, although transcriptional and translational regions from the host species are particularly useful. Expression in a microbial host cell can be accomplished in an induced or constitutive fashion. Induced expression can be accomplished by inducing the activity of a regulatable promoter operably linked to the gene of interest, while constitutive expression can be achieved by the use of a constitutive promoter operably linked to the gene of interest. As an example, when the host cell is yeast, transcriptional and translational regions functional in yeast cells are provided, particularly from the host species (e.g., see Patent Publication No. US-2006-0115881-A1, corresponding to PCT Publication No. WO 2006/052870 for preferred transcriptional initiation regulatory regions for use in Yarrowia lipolytica). Any one of a number of regulatory sequences can be used, depending upon whether constitutive or induced transcription is desired, the efficiency of the promoter in expressing the ORF of interest, the ease of construction and the like.

The termination region can be derived from the 3′ region of the gene from which the initiation region was obtained or from a different gene. A large number of termination regions are known and function satisfactorily in a variety of hosts (when utilized both in the same and different genera and species from where they were derived). The termination region usually is selected more as a matter of convenience rather than because of any particular property. Termination control regions may also be derived from various genes native to the preferred hosts. In alternate embodiments, the 3′-region can also be synthetic, as one of skill in the art can utilize available information to design and synthesize a 3′-region sequence that functions as a transcription terminator. A termination region may be unnecessary, but it is highly preferred.

As one of skill in the art is aware, merely inserting an isolated polynucleotide into a cloning vector does not ensure that it will be successfully expressed at the level needed. In response to the need for a high expression rate, many specialized expression vectors have been created by manipulating a number of different genetic elements that control aspects of transcription, translation, protein stability, oxygen limitation and secretion from the microbial host cell. More specifically, some of the molecular features that have been manipulated to control gene expression include: the nature of the relevant transcriptional promoter and terminator sequences; the number of copies of the cloned gene (wherein additional copies may be cloned within a single expression construct and/or additional copies may be introduced into the host cell by increasing the plasmid copy number or by multiple integration of the cloned gene into the genome); whether the gene is plasmid-borne or integrated into the genome of the host cell; the final cellular location of the synthesized foreign protein; the efficiency of translation and correct folding of the protein in the host organism; the intrinsic stability of the mRNA and protein of the cloned gene within the host cell; and, the codon usage within the cloned gene, such that its frequency approaches the frequency of preferred codon usage of the host cell. Each of these types of modifications are encompassed in the present disclosure, as means to further optimize expression of ACBP.

Once a DNA cassette that is suitable for expression in an appropriate microbial host cell has been obtained (e.g., a chimeric gene comprising a promoter, ORF and terminator or a gene knock-out construct), it is placed in a plasmid vector capable of autonomous replication in a host cell, or it is directly integrated into the genome of the host cell. Integration of expression cassettes can occur randomly within the host genome or can be targeted through the use of constructs containing regions of homology with the host genome sufficient to target recombination within the host locus. Where constructs are targeted to an endogenous locus, all or some of the transcriptional and translational regulatory regions can be provided by the endogenous locus.

Where two or more isolated polynucleotides are expressed from separate replicating vectors, it is desirable that each vector has a different means of selection and should lack homology to the other construct(s) to maintain stable expression and prevent reassortment of elements among constructs. Judicious choice of regulatory regions, selection means and method of propagation of the introduced construct(s) can be experimentally determined so that all introduced genes are expressed at the necessary levels to provide for synthesis of the desired products.

Constructs comprising the isolated polynucleotide(s) of interest may be introduced into a microbial host cell by any standard technique. These techniques include transformation (e.g., lithium acetate transformation [Methods in Enzymology, 194:186-187 (1991)]), protoplast transformation, bolistic impact, electroporation, microinjection, or any other method that introduces the gene(s) of interest into the host cell.

For convenience, a host cell that has been manipulated by any method to take up a DNA sequence (e.g., an expression cassette) will be referred to as “transformed”, “transformant” or “recombinant” herein. The transformed host will have at least one copy of the expression construct and may have two or more, depending upon whether the expression cassette is integrated into the genome or is present on an extrachromosomal element having multiple copy numbers.

The transformed host cell can be identified by various selection techniques, as described in U.S. Pat. No. 7,238,482, U.S. Pat. No. 7,259,255 and PCT Publication No. WO 2006/052870.

Following transformation, fatty acid substrates suitable for binding to ACBP may be produced by the host either naturally or transgenically, or they may be provided exogenously. In preferred embodiments, however, the oleaginous microbial organism possesses the ability to produce PUFAs, either naturally or via techniques of genetic engineering. Frequently, it will be expected that the microbial organism will comprise heterologous genes encoding a functional PUFA biosynthetic pathway (although this should not be construed as a limitation herein).

If the desired PUFAs (or desired lipid profile) are not endogenously produced by the microbial organism, one skilled in the art will be familiar with the considerations and techniques necessary to introduce an expression cassette(s) encoding appropriate enzymes for PUFA biosynthesis into the microbial organism of choice. Although these issues are not elaborated in detail herein, numerous teachings are provided in the literature; and, some illustrative references are provided as follows, although these should not be construed as limiting: WO 98/46763; WO 98/46764; WO 98/46765; WO 99/64616; WO 02/077213; WO 03/093482; WO 04/057001; WO 04/090123; WO 04/087902; U.S. Pat. No. 6,140,486; U.S. Pat. No. 6,459,018; U.S. Pat. No. 6,136,574; U.S. Pat. No. 7,238,482; U.S. 03/0172399; U.S. 04/0172682; U.S. 04/098762; U.S. 04/0111763; U.S. 04/0053379; U.S. 04/0049805; U.S. 04/0237139; U.S. 04/0172682; Beaudoin F. et al., PNAS USA, 97(12):6421-6426 (2000); Dyer, J. M. et al., Appl. Envi. Microbiol., 59:224-230 (2002); Domergue, F. et al. Eur. J. Biochem. 269:4105A4113 (2002); Qi, B. et al., Nature Biotech. 22:739-745 (2004); and Abbadi et al., The Plant Cell, 16:2734-2748 (2004)).

Briefly, however, a variety of ω-3/ω-6 PUFA products can be produced (prior to their transfer to TAGs), depending on the fatty acid substrate and the particular genes of the ω-3/ω-6 fatty acid biosynthetic pathway that are present in (or transformed into) the microbial cell. As such, production of the desired fatty acid product can occur directly (wherein the fatty acid substrate is converted directly into the desired fatty acid product without any intermediate steps or pathway intermediates) or indirectly (wherein multiple genes encoding the PUFA biosynthetic pathway may be used in combination, such that a series of reactions occur to produce a desired PUFA). Specifically, for example, it may be desirable to transform an oleaginous yeast with expression cassette(s) comprising Δ9 elongase, Δ8 desaturase, Δ5 desaturase and Δ17 desaturase for the overproduction of EPA. As is well known to one skilled in the art, various other combinations of the following enzymatic activities may be useful to express in an oleaginous organism: Δ6 desaturases, C_{18/20} elongases, Δ5 desaturases, Δ17 desaturases, Δ15 desaturases, Δ9 desaturases, Δ12 desaturases, C_{14/16} elongases, C_{16/18} elongases, Δ9 elongases, Δ8 desaturases, Δ4 desaturases and C_{20/22} elongases (see FIG. 1). The particular genes included within a particular expression cassette will depend on the oleaginous organism (and its PUFA profile and/or desaturase/elongase profile), the availability of substrate and the desired end product(s).

One skilled in the art will be able to identify various candidate genes encoding each of the enzymes desired for ω-3/ω-6 fatty acid biosynthesis, based on publicly available literature (e.g., GenBank), the patent literature, and experimental analysis of organisms having the ability to produce PUFAs. Useful desaturase and elongase sequences may be derived from any source, e.g., isolated from a natural source (from bacteria, algae, fungi, plants, animals, etc.), produced via a semi-synthetic route or synthesized de novo. Although the particular source of the desaturase and elongase genes introduced into the host is not critical, considerations for choosing a specific polypeptide having desaturase or elongase activity include: 1) the substrate specificity of the polypeptide; 2) whether the polypeptide or a component thereof is a rate-limiting enzyme; 3) whether the desaturase or elongase is essential for synthesis of a desired PUFA; 4) co-factors required by the polypeptide; and/or, 5) whether the polypeptide was modified after its production (e.g., by a kinase or a prenyltransferase). The expressed polypeptide preferably has parameters compatible with the biochemical environment of its location in the host cell (see U.S. Pat. No. 7,238,482 for additional details).

In additional embodiments, it will also be useful to consider the conversion efficiency of each particular desaturase and/or elongase. More specifically, since each enzyme rarely functions with 100% efficiency to convert substrate to product, the final lipid profile of unpurified oils produced in a host cell will typically be a mixture of various PUFAs consisting of the desired ω-3/ω-6 fatty acid, as well as various upstream intermediary PUFAs. Thus, each enzyme's conversion efficiency is also a variable to consider, when optimizing biosynthesis of a desired fatty acid.

Microbial host cells for suitable for manipulation of the total lipid content and/or fatty acid composition as disclosed herein may include hosts that grow on a variety of feedstocks, including simple or complex carbohydrates, fatty acids, organic acids, oils, glycerol and alcohols, and/or hydrocarbons over a wide range of temperature and pH values. Based on the needs of the Applicants' Assignee, the methods have been developed for use in oleaginous yeast (and in particular Yarrowia lipolytica); however, it is contemplated that because transcription, translation and the protein biosynthetic apparatus are highly conserved, any bacteria, yeast, algae, euglenoid, stramenopiles and/or fungus will be a suitable oleaginous microbe.

Preferred microbial hosts, however, are oleaginous organisms, such as oleaginous yeasts. These organisms are naturally capable of oil synthesis and accumulation, wherein the oil can comprise greater than about 25% of the cellular dry weight, more preferably greater than about 30% of the cellular dry weight, and most preferably greater than about 40% of the cellular dry weight. Genera typically identified as oleaginous yeast include, but are not limited to: Yarrowia, Candida, Rhodotorula, Rhodosporidium, Cryptococcus, Trichosporon and Lipomyces. More specifically, illustrative oil-synthesizing yeasts include: Rhodosporidium toruloides, Lipomyces starkeyii, L. lipoferus, Candida revkaufi, C. pulcherrima, C. tropicalis, C. utilis, Trichosporon pullans, T. cutaneum, Rhodotorula glutinus, R. graminis, and Yarrowia lipolytica (formerly classified as Candida lipolytica). In alternate embodiments, oil biosynthesis may be genetically engineered such that the microbial host cell (e.g., a yeast) can produce more than 25% oil of the cellular dry weight, and thereby be considered oleaginous.

Most preferred is the oleaginous yeast Yarrowia lipolytica; and, in a further embodiment, most preferred are the Y. lipolytica strains designated as ATCC #20362, ATCC #8862, ATCC #18944, ATCC #76982 and/or LGAM S(7)1 (Papanikolaou S., and Aggelis G., Bioresour. Technol., 82(1):43-9 (2002)).

Specific teachings applicable for transformation of oleaginous yeasts (i.e., Yarrowia lipolytica) include U.S. Pat. No. 4,880,741 and U.S. Pat. No. 5,071,764 and Chen, D. C. et al. (Appl. Microbiol. Biotechnol., 48(2):232-235 (1997)). Specific teachings applicable for engineering GLA, ARA, EPA and DHA production in Y. lipolytica are provided in U.S. patent application Ser. No. 11/198,975 (PCT Publication No. WO 2006/033723), U.S. patent application Ser. No. 11/264,784 (PCT Publication No. WO 2006/055322), U.S. patent application Ser. No. 11/265,761 (PCT Publication No. WO 2006/052870) and U.S. patent application Ser. No. 11/264,737 (PCT Publication No. WO 2006/052871), respectively.

The preferred method of expressing isolated polynucleotide(s) in this yeast is by integration of linear DNA into the genome of the host; and, integration into multiple locations within the genome can be particularly useful when high level expression of genes are desired [e.g., in the Ura3 locus (GenBank Accession No. AJ306421), the Leu2 gene locus (GenBank Accession No. AF260230), the Lys5 gene locus (GenBank Accession No. M34929), the Aco2 gene locus (GenBank Accession No. AJ001300), the Pox3 gene locus (Pox3: GenBank Accession No. XP_{—}503244; or, Aco3: GenBank Accession No. AJ001301), the Δ12 desaturase gene locus (U.S. Pat. No. 7,214,491), the Lip1 gene locus (GenBank Accession No. Z50020), the Lip2 gene locus (GenBank Accession No. AJ012632), the SCP2 gene locus (GenBank Accession No. AJ431362), the Pex3 gene locus (GenBank Accession No. CAG78565), the Pex16 gene locus (GenBank Accession No. CAG79622) and/or the Pex10 gene locus (GenBank Accession No. CAG81606)].

Preferred selection methods for use in Yarrowia lipolytica are resistance to kanamycin, hygromycin and the amino glycoside G418, as well as ability to grow on media lacking uracil, leucine, lysine, tryptophan or histidine. In alternate embodiments, 5-fluoroorotic acid (5-fluorouracil-6-carboxylic acid monohydrate; “5-FOA”) is used for selection of yeast Ura⁻ mutants (U.S. Pat. Pub. No. 2009-0093543-A1), or a native acetohydroxyacid synthase (or acetolactate synthase; E.C. 4.1.3.18) that confers sulfonyl urea herbicide resistance (PCT Publication No. WO 2006/052870) is utilized for selection of transformants.

In yet another aspect, the invention concerns a method for increasing the total lipid content in a Yarrowia sp., comprising:
a) providing Yarrowia sp., comprising:
   i) a recombinant construct comprising at least one isolated polynucleotide comprising a nucleic acid sequence encoding an acyl-CoA binding protein, said protein selected from the group consisting of:
      (a) a protein consisting essentially of the sequence set forth in SEQ ID NO:2; and
      (b) a protein comprising the following amino acid sequence motifs: SEQ ID NO:36, SEQ ID NO:37 and SEQ ID NO:38;
   wherein said isolated polynucleotide is operably linked to at least one regulatory sequence; and
   ii) a source of fatty acids;
b) growing the Yarrowia sp. of step (a) under conditions whereby the expression of the acyl-CoA binding protein results in an increased total lipid content of at least 5% when compared to the total lipid content of a Yarrowia sp. lacking said recombinant construct; and,
c) optionally recovering the total lipids of step (b).

Preferably, expression of ACBP results in an increased total lipid content of at least 10% when compared to the lipid content of a Yarrowia sp. lacking said recombinant construct, more preferably an increased lipid content of at least 15%, and most preferably an increased lipid content of at least 20%. Higher percent increases in the lipid content may additionally require expression of at least one DAG AT sequence, in addition to the ACBP, wherein the DAG AT is preferably selected from the group consisting of DGAT1 (e.g., SEQ ID NO:4), DGAT2 (e.g., SEQ ID NOs:6, 8, 9, 10 and 11) or a combination thereof.

In some embodiments, the fatty acids of step (b) are endogenously produced, preferably by expression of a functional ω-3/ω-6 fatty acid biosynthetic pathway including at least one desaturase enzyme (e.g., selected from the group consisting of Δ4 desaturase, Δ5 desaturase, Δ9 desaturase, Δ12 desaturase, Δ15 desaturase, Δ17 desaturase, Δ8 desaturase). Modification of the expression of ACBP in these Yarrowia sp. will result in an altered rate of desaturation by the desaturase, thereby resulting in an altered transfer of the synthesized fatty acids to TAG and an altered fatty acid composition.

Other suitable microbial hosts include oleaginous bacteria, algae, euglenoids, stramenopiles and other fungi; and, within this broad group of microbial hosts, of particular interest are microorganisms that synthesize ω-3/ω-6 fatty acids (or those that can be genetically engineered for this purpose [e.g., other yeast such as Saccharomyces cerevisiae]). Thus, for example, overexpression of a native ACBP in Mortierella alpina (which is commercially used for production of ARA) should yield a transformant organism having increased total lipid content. The method of transformation of M. alpina is described by Mackenzie et al. (Appl. Environ. Microbiol., 66:4655 (2000)). Similarly, methods for transformation of Thraustochytriales microorganisms (e.g., Thraustochytrium, Schizochytrium) are disclosed in U.S. Pat. No. 7,001,772.

Irrespective of the host selected for modifying the total lipid content and/or composition as described herein, multiple transformants must be screened in order to obtain a strain displaying the desired expression level and pattern. Such screening may be accomplished by Southern analysis of DNA blots (Southern, J. Mol. Biol., 98:503 (1975)), Northern analysis of mRNA expression (Kroczek, J. Chromatogr. Biomed. Appl., 618(1-2):133-145 (1993)), Western and/or Elisa analyses of protein expression, phenotypic analysis or GC analysis of the PUFA products.

Also described herein are oleaginous microbial organisms produced by the methods described herein. This therefore includes oleaginous bacteria, algae, moss, euglenoids, stramenopiles fungi and yeast, comprising in their genome a recombinant ACBP construct. Additionally, lipids and oils obtained from these oleaginous organisms, products obtained from the processing of the lipids and oil, use of these lipids and oil in foods, animal feeds or industrial applications and/or use of the by-products in foods or animal feeds are also described.

The transformed microbial host cell is grown under conditions that optimize expression of ACBP and produce the greatest and most economical yield of fatty acids, preferably comprising an optimal PUFA composition. In general, media conditions that may be optimized include the type and amount of carbon source, the type and amount of nitrogen source, the carbon-to-nitrogen ratio, the amount of different mineral ions, the oxygen level, growth temperature, pH, length of the biomass production phase, length of the oil accumulation phase and the time and method of cell harvest. Microorganisms of interest, such as oleaginous yeast (e.g., Yarrowia lipolytica) are generally grown in complex media (e.g., yeast extract-peptone-dextrose broth (YPD)) or a defined minimal media that lacks a component necessary for growth and thereby forces selection of the desired expression cassettes (e.g., Yeast Nitrogen Base (DIFCO Laboratories, Detroit, Mich.)).

Fermentation media used herein must contain a suitable carbon source. Suitable carbon sources are taught in U.S. Pat. No. 7,238,482. Although it is contemplated that the source of carbon utilized may encompass a wide variety of carbon-containing sources, preferred carbon sources are sugars (e.g., glucose), glycerol, and/or fatty acids.

Nitrogen may be supplied from an inorganic (e.g., (NH₄)₂SO₄) or organic (e.g., urea or glutamate) source. In addition to appropriate carbon and nitrogen sources, the fermentation media must also contain suitable minerals, salts, cofactors, buffers, vitamins and other components known to those skilled in the art suitable for the growth of the oleaginous host and promotion of the enzymatic pathways necessary for PUFA production. Particular attention is given to several metal ions (e.g., Fe⁺², Cu⁺², Mn⁺², Co⁺², Zn⁺², Mg⁺²) that promote synthesis of lipids and PUFAs (Nakahara, T. et al., Ind. Appl. Single Cell Oils, D. J. Kyle and R. Colin, eds. pp 61-97 (1992)).

Preferred growth media are common commercially prepared media, such as Yeast Nitrogen Base (DIFCO Laboratories, Detroit, Mich.). Other defined or synthetic growth media may also be used and the appropriate medium for growth of the transformant host cells will be known by one skilled in the art of microbiology or fermentation science. A suitable pH range for the fermentation is typically between about pH 4.0 to pH 8.0, wherein pH 5.5 to pH 7.5 is preferred as the range for the initial growth conditions. The fermentation may be conducted under aerobic or anaerobic conditions, wherein microaerobic conditions are preferred.

Typically, accumulation of high levels of PUFAs in oleaginous yeast cells requires a two-stage process, since the metabolic state must be “balanced” between growth and synthesis/storage of fats. Thus, most preferably, a two-stage fermentation process is necessary for the production of PUFAs in oleaginous yeast (e.g., Yarrowia lipolytica). This approach is described in U.S. Pat. No. 7,238,482, as are various suitable fermentation process designs (i.e., batch, fed-batch and continuous) and considerations during growth.

PUFAs may be found in the host microorganisms as free fatty acids or in esterified forms such as acylglycerols, phospholipids, sulfolipids or glycolipids, and may be extracted from the host cells through a variety of means well-known in the art. One review of extraction techniques, quality analysis and acceptability standards for yeast lipids is that of Z. Jacobs (Critical Reviews in Biotechnology, 12(5/6):463-491 (1992)). A brief review of downstream processing is also available by A. Singh and O. Ward (Adv. Appl. Microbiol., 45:271-312 (1997)).

In general, means for the purification of PUFAs may include extraction (e.g., U.S. Pat. No. 6,797,303 and U.S. Pat. No. 5,648,564) with organic solvents, sonication, supercritical fluid extraction (e.g., using carbon dioxide), saponification and physical means such as presses, or combinations thereof. One is referred to the teachings of U.S. Pat. No. 7,238,482 for additional details.

There are a plethora of food and feed products, incorporating ω-3 and/or ω-6 fatty acids (particularly e.g., ALA, GLA, ARA, EPA, DPA and DHA). It is contemplated that the microbial biomass comprising long-chain PUFAs, partially purified microbial biomass comprising PUFAs, purified microbial oil comprising PUFAs, and/or purified PUFAs will function in food and feed products to impart the health benefits of current formulations. More specifically, oils containing ω-3 and/or ω-6 fatty acids will be suitable for use in a variety of food and feed products including, but not limited to: food analogs, meat products, cereal products, baked foods, snack foods and dairy products (see Patent Publication No. US-2006-0094092 for details).

Additionally, the present compositions may be used in formulations to impart health benefit in medical foods including medical nutritionals, dietary supplements, infant formula as well as pharmaceutical products. One of skill in the art of food processing and food formulation will understand how the amount and composition of the present oils may be added to the food or feed product. Such an amount will be referred to herein as an “effective” amount and will depend on the food or feed product, the diet that the product is intended to supplement or the medical condition that the medical food or medical nutritional is intended to correct or treat.

### EXAMPLES

The present invention is further described in the following Examples, which illustrate reductions to practice of the invention but do not completely define all of its possible variations.

### General Methods

Standard recombinant DNA and molecular cloning techniques used in the Examples are well known in the art and are described by:

1) Sambrook, J., Fritsch, E. F. and Maniatis, T., Molecular Cloning: A Laboratory Manual; Cold Spring Harbor Laboratory: Cold Spring Harbor, N.Y. (1989) (Maniatis); 2) T. J. Silhavy, M. L. Bennan, and L. W. Enquist, Experiments with Gene Fusions; Cold Spring Harbor Laboratory: Cold Spring Harbor, N.Y. (1984); and, 3) Ausubel, F. M. et al., Current Protocols in Molecular Biology, published by Greene Publishing Assoc. and Wiley-Interscience, Hoboken, N.J. (1987).

Materials and methods suitable for the maintenance and growth of microbial cultures are well known in the art. Techniques suitable for use in the following examples may be found as set out in Manual of Methods for General Bacteriology (Phillipp Gerhardt, R. G. E. Murray, Ralph N. Costilow, Eugene W. Nester, Willis A. Wood, Noel R. Krieg and G. Briggs Phillips, Eds), American Society for Microbiology: Washington, D.C. (1994)); or by Thomas D. Brock in Biotechnology: A Textbook of Industrial Microbiology, 2^{nd} ed., Sinauer Associates: Sunderland, Mass. (1989). All reagents, restriction enzymes and materials used for the growth and maintenance of microbial cells were obtained from Aldrich Chemicals (Milwaukee, Wis.), DIFCO Laboratories (Detroit, Mich.), GIBCO/BRL (Gaithersburg, Md.), or Sigma Chemical Company (St. Louis, Mo.), unless otherwise specified. E. coli strains were typically grown at 37° C. on Luria Bertani [“LB”] plates.

General molecular cloning was performed according to standard methods (Sambrook et al., supra). DNA sequence was generated on an ABI Automatic sequencer using dye terminator technology (U.S. Pat. No. 5,366,860; EP 272,007) using a combination of vector and insert-specific primers. Sequence editing was performed in Sequencher (Gene Codes Corporation, Ann Arbor, Mich.). All sequences represent coverage at least two times in both directions. Comparisons of genetic sequences were accomplished using DNASTAR software (DNASTAR Inc., Madison, Wis.).

The meaning of abbreviations is as follows: “sec” means second(s), “min” means minute(s), “hr” means hour(s), “d” means day(s), “μL” means microliter(s), “mL” means milliliter(s), “L” means liter(s), “μM” means micromolar, “mM” means millimolar, “M” means molar, “mmol” means millimole(s), “μmole” mean micromole(s), “g” means gram(s), “μg” means microgram(s), “ng” means nanogram(s), “U” means unit(s), “bp” means base pair(s) and “kB” means kilobase(s).

### Nomenclature for Expression Cassettes:

The structure of an expression cassette will be represented by a simple notation system of “X::Y::Z”, wherein X describes the promoter fragment, Y describes the gene fragment, and Z describes the terminator fragment, which are all operably linked to one another.

Transformation and Cultivation of Yarrowia lipolytica:

Yarrowia lipolytica strains with ATCC Accession Nos. #20362, #76982 and #90812 were purchased from the American Type Culture Collection (Rockville, Md.). Yarrowia lipolytica strains were typically grown at 28-30° C. in several media, according to the recipes shown below. Agar plates were prepared as required by addition of 20 g/L agar to each liquid media, according to standard methodology.
YPD agar medium (per liter): 10 g of yeast extract [Difco]; 20 g of Bacto peptone [Difco]; and 20 g of glucose.
Basic Minimal Media (MM) (per liter): 20 g glucose; 1.7 g yeast nitrogen base without amino acids; 1.0 g proline; and pH 6.1 (not adjusted).
Minimal Media+5-Fluoroorotic Acid (MM+5-FOA) (per liter): 20 g glucose; 6.7 g Yeast Nitrogen base; 75 mg uracil; 75 mg uridine; and appropriate amount of FOA (Zymo Research Corp., Orange, Calif.), based on FOA activity testing against a range of concentrations from 100 mg/L to 1000 mg/L (since variation occurs within each batch received from the supplier).
High Glucose Media (HGM) (per liter): 80 glucose; 2.58 g KH₂PO₄; and 5.36 g K₂HPO₄; pH 7.5 (do not need to adjust).
Fermentation medium (FM) (per liter): 6.70 g/L Yeast nitrogen base; 6.00 g KH₂PO₄; 2.00 g K₂HPO₄; 1.50 g MgSO₄.7H₂O; 20 g glucose; and 5.00 g Yeast extract (BBL).

Transformation of Yarrowia lipolytica was performed according to the method of Chen, D. C. et al. (Appl. Microbiol. Biotechnol., 48(2):232-235 (1997)), unless otherwise noted. Briefly, Yarrowia was streaked onto a YPD plate and grown at 30° C. for approximately 18 hr. Several large loopfuls of cells were scraped from the plate and resuspended in 1 mL of transformation buffer, comprising: 2.25 mL of 50% PEG, average MW 3350; 0.125 mL of 2 M lithium acetate, pH 6.0; 0.125 mL of 2 M DTT; and (optionally) 50 μg sheared salmon sperm DNA. Then, approximately 500 ng of linearized plasmid DNA (or 100 ng circular plasmid) was incubated in 100 μL of resuspended cells, and maintained at 39° C. for 1 hr with vortex mixing at 15 min intervals. The cells were plated onto selection media plates and maintained at 30° C. for 2 to 3 days.

Fatty Acid Analysis of Yarrowia lipolytica:

Unless otherwise stated, for fatty acid analysis cells were collected by centrifugation and lipids were extracted as described in Bligh, E. G. & Dyer, W. J. (Can. J. Biochem. Physiol., 37:911-917 (1959)). Fatty acid methyl esters [“FAMEs”] were prepared by transesterification of the lipid extract with sodium methoxide (Roughan, G. and Nishida I., Arch Biochem Biophys., 276(1):38-46 (1990)) and subsequently analyzed with a Hewlett-Packard 6890 GC fitted with a 30 m×0.25 mm (i.d.) HP-INNOWAX (Hewlett-Packard) column. The oven temperature was from 170° C. (25 min hold) to 185° C. at 3.5° C./min.

For direct base transesterification, Yarrowia culture (3 mL) was harvested, washed once in distilled water, and dried under vacuum in a Speed-Vac for 5-10 min. Sodium methoxide (100 μL of 1%) was added to the sample, and then the sample was vortexed and rocked for 20 min. After adding 3 drops of 1 M NaCl and 400 μL hexane, the sample was vortexed and spun. The upper layer was removed and analyzed by GC as described above.

Construction of Yarrowia lipolytica Strains Y4036U And Y4305U1:

Strain Y4036U, derived from Yarrowia lipolytica ATCC #20362, is capable of producing DGLA in the total lipids via expression of a Δ9 elongase/Δ8 desaturase pathway. Y. lipolytica strain Y4036U was used as the host in Example 1, infra.

Strain Y4305U1, derived from Yarrowia lipolytica strain Y4036U, is capable of producing about 53.2% EPA relative to the total lipids via expression of a Δ9 elongase/Δ8 desaturase pathway. Y. lipolytica strain Y4305U1 was used as the host in Example 2, infra.

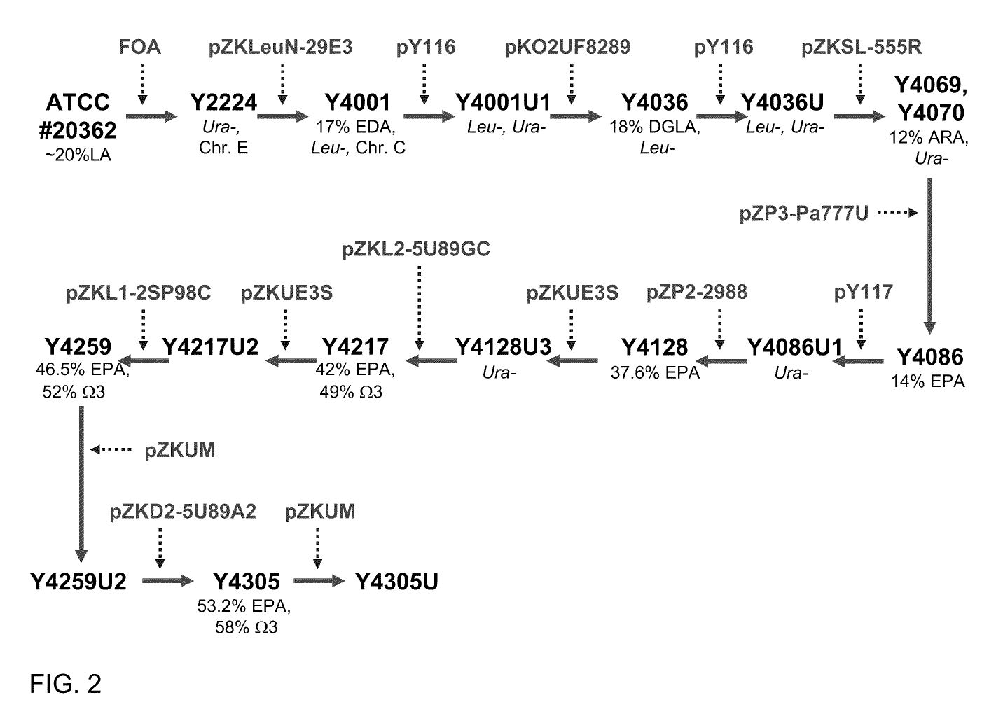

Briefly, as diagrammed in FIG. 2, strain Y4305U1 was derived from Yarrowia lipolytica ATCC #20362 via construction of strain Y2224 (a FOA resistant mutant from an autonomous mutation of the Ura3 gene of wildtype Yarrowia strain ATCC #20362), strain Y4001 (producing 17% EDA with a Leu-phenotype), strain Y4001U1 (Leu- and Ura-), strain Y4036 (producing 18% DGLA with a Leu-phenotype), strain Y4036U (Leu- and Ura-), strain Y4070 (producing 12% ARA with a Ura-phenotype), strain Y4086 (producing 14% EPA), strain Y4086U1 (Ura-), strain Y4128 (producing 37% EPA; deposited with the American Type Culture Collection on Aug. 23, 2007, bearing the designation ATCC PTA-8614), strain Y4128U3 (Ura-), strain Y4217 (producing 42% EPA), strain Y4217U2 (Ura-), strain Y4259 (producing 46.5% EPA), strain Y4259U2 (Ura-) and strain Y4305 (producing 53.2% EPA relative to the total TFAs). Further details regarding the construction of strains Y2224, Y4001, Y4001U, Y4036, Y4036U, Y4070, Y4086, Y4086U1, Y4128, Y4128U3, Y4217, Y4217U2, Y4259, Y4259U2, Y4305 and Y4305U3 are described in the General Methods of U.S. Pat. App. Pub. No. 2008-0254191 and in Examples 1-3 of U.S. Pat. App. Pub. No. 2009-0093543, hereby incorporated herein by reference.

The final genotype of strain Y4036U with respect to wild type Yarrowia lipolytica ATCC #20362 was Ura3-, YAT1::ME3S::Pex16, EXP1::EgD9eS::Lip1, FBAINm::EgD9eS::Lip2, GPAT::EgD9e::Lip2, FBAINm::EgD8M::Pex20, EXP1::EgD8M::Pex16, GPD::FmD12::Pex20, YAT1::FmD12::OCT (wherein FmD12 is a Fusarium moniliforme A12 desaturase gene [U.S. Pat. No. 7,504,259]; ME3S is a codon-optimized C_{16/18} elongase gene, derived from Mortierella alpina [U.S. Pat. No. 7,470,532]; EgD9e is a Euglena gracilis Δ9 elongase gene [Int'l. App. Pub. No. WO 2007/061742]; EgD9eS is a codon-optimized A9 elongase gene, derived from Euglena gracilis [Int'l. App. Pub. No. WO 2007/061742]; and, EgD8M is a synthetic mutant A8 desaturase [Int'l. App. Pub. No. WO 2008/073271], derived from Euglena gracilis [U.S. Pat. No. 7,256,033]).

The complete lipid profile of strain Y4305 was as follows, wherein the concentration of each fatty acid is expressed as a weight percentable of the TFAs: 16:0 (2.8%), 16:1 (0.7%), 18:0 (1.3%), 18:1 (4.9%), 18:2 (17.6%), ALA (2.3%), EDA (3.4%), DGLA (2.0%), ARA (0.6%), ETA (1.7%), and EPA (53.2%). The total lipid content of cells [“TFAs % DCW”] was 27.5.

The final genotype of strain Y4305 with respect to wild type Yarrowia lipolytica ATCC #20362 was SCP2- (YALI0E01298g), YALI0C18711g-, Pex10-, YALI0F24167g-, unknown 1-, unknown 3-, unknown 8-, GPD::FmD12::Pex20, YAT1::FmD12::OCT, GPM/FBAIN::FmD12S::OCT, EXP1::FmD12S::Aco, YAT1::FmD12S::Lip2, YAT1::ME3S::Pex16, EXP1::ME3S::Pex20 (3 copies), GPAT::EgD9e::Lip2, EXP1::EgD9eS::Lip1, FBAlNm::EgD9eS::Lip2, FBA::EgD9eS::Pex20, GPD::EgD9eS::Lip2, YAT1::EgD9eS::Lip2, YAT1::E389D9eS::OCT, FBAINm::EgD8M::Pex20, FBAIN::EgD8M::Lip1 (2 copies), EXP1::EgD8M::Pex16, GPDIN::EgD8M::Lip1, YAT1::EgD8M::Aco, FBAIN::EgD5::Aco, EXP1::EgD5S::Pex20, YAT1::EgD5S::Aco, EXP1::EgD5S::ACO, YAT1::RD5S::OCT, YAT1::PaD17S::Lip1, EXP1::PaD17::Pex16, FBAINm::PaD17::Aco, YAT1::YICPT1::ACO, GPD::YICPT1::ACO (wherein FmD12 is a Fusarium moniliforme A12 desaturase gene [U.S. Pat. No. 7,504,259]; FmD12S is a codon-optimized A12 desaturase gene, derived from Fusarium moniliforme [U.S. Pat. No. 7,504,259]; ME3S is a codon-optimized C_{16/18} elongase gene, derived from Mortierella alpina [U.S. Pat. No. 7,470,532]; EgD9e is a Euglena gracilis Δ9 elongase gene [Int'l. App. Pub. No. WO 2007/061742]; EgD9eS is a codon-optimized A9 elongase gene, derived from Euglena gracilis [Int'l. App. Pub. No. WO 2007/061742]; E389D9eS is a codon-optimized A9 elongase gene, derived from Eutreptiella sp. CCMP389 [Int'l. App. Pub. No. WO 2007/061742]; EgD8M is a synthetic mutant Δ8 desaturase [Int'l. App. Pub. No. WO 2008/073271], derived from Euglena gracilis [U.S. Pat. No. 7,256,033]; EgD5 is a Euglena gracilis Δ5 desaturase [U.S. Pat. App. Pub. No. 2007-0292924-A1]; EgD5S is a codon-optimized A5 desaturase gene, derived from Euglena gracilis [U.S. Pat. App. Pub. No. 2007-0292924]; RD5S is a codon-optimized A5 desaturase, derived from Peridinium sp. CCMP626 [U.S. Pat. App. Pub. No. 2007-0271632]; PaD17 is a Pythium aphanidermatum Δ17 desaturase [Int'l. App. Pub. No. WO 2008/054565]; PaD17S is a codon-optimized Δ17 desaturase, derived from Pythium aphanidermatum [Int'l. App. Pub. No. WO 2008/054565]; and, YICPT1 is a Yarrowia lipolytica diacylglycerol cholinephosphotransferase [Int'l. App. Pub. No. WO 2006/052870]).

Strains Y4305U1, Y4305U2 and Y4305U3 ((collectively, Y4305U) were generated by integrating a Ura3 mutant gene into the Ura3 gene of strain Y4305.

### Example 1

### Chromosomal Deletion of YL ACBP Reduced Lipid Accumulation in Yarrowia lipolytica Strain Y4036U

The present Example describes the construction of pYPS161-ACBP, a vector used to disrupt the chromosomal acb1 gene (i.e., YL ACBP, set forth as SEQ ID NO:1) from the DGLA-producing Yarrowia strain Y4036U. Transformation of Y. lipolytica strain Y4036U with the acb1 knockout construct resulted in creation of strain Y4036U Δacb1. The effect of the acb1 knockout on total oil and DGLA level was determined and compared. Specifically, knockout of acb1 resulted in a reduced amount of total lipid in the cell.

Construct pYPS161-ACBP

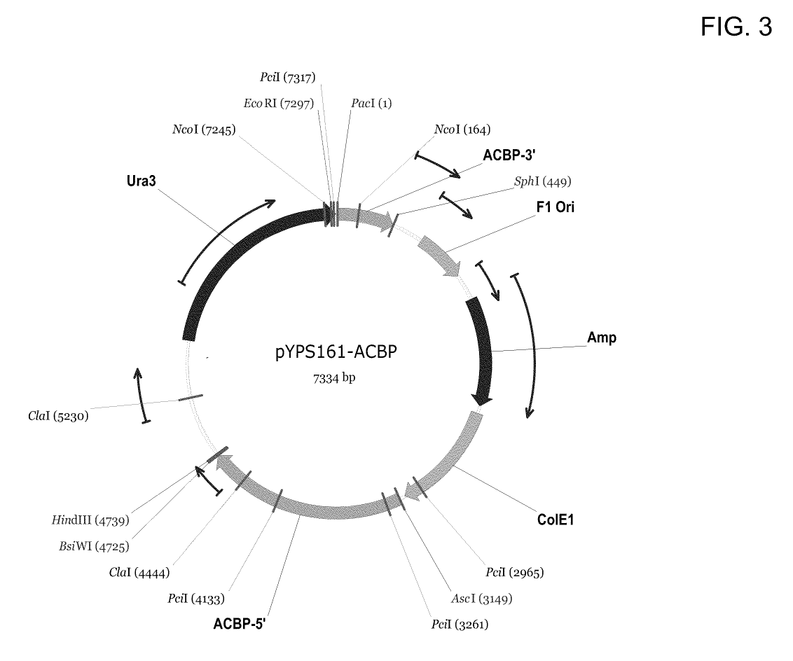

Plasmid pYPS161-ACBP (FIG. 3) was derived from plasmid pYPS161 (SEQ ID NO:12, comprising a Yarrowia URA3 gene, ColE1 plasmid origin of replication, and ampicillin-resistance gene for selection in E. coli) and constructed to generate an acb1 deletion strain of Yarrowia lipolytica. The pYPS161-ACBP plasmid thereby contains the following components:

TABLE 3
Description of Plasmid pYPS161-ACBP (SEQ ID NO: 13)

| RE Sites And | |
|---|---|
| Nucleotides | |
| Within SEQ ID | Description Of Fragment And |
| NO: 13 | Chimeric Gene Components |
| AscI/BsiWI | 1576 bp 5′ promoter region of Yarrowia lipolytica ACB1 gene |
| (3149-4725) | (ORF YALI0E23185g within the public Y. lipolytica protein |
| | database of the “Yeast project Genolevures” (Center for |
| | Bioinformatics, LaBRI, Talence Cedex, France) |
| PacI/SphI | 448 bp 3′ terminator region of Yarrowia lipolytica ACB1 gene |
| (1-449) | (ORF YALI0E23185g, supra) |
| Sa/I/EcoRI | Yarrowia URA3 gene (GenBank Accession No. AJ306421) |
| (5678-7297) | |
| 2222-3102 | ColE1 plasmid origin of replication |
| 1304-2164 | ampicillin-resistance gene (Amp^{R}) for selection in E. coli |
| 696-1096 | E. coli f1 origin of replication |

Generation of Yarrowia lipotytica Knockout Strain Y4036U Δacb1

Standard protocols were used to transform Yarrowia lipolytica strain Y4036U (see General Methods) with the purified 4.6 kB AscI/SphI fragment of ACB1 knockout construct pYPS161-ACBP. The fragment contained the URA3 gene as a selectable marker to facilitate selection of transformants on media plates lacking uracil.

To screen for the acb1 deleted mutant, colony PCR was performed using Taq polymerase (Invitrogen; Carlsbad, Calif.), and the PCR primers ACBPFii (SEQ ID NO:14) and ACBPRii (SEQ ID NO:15). This set of primers was designed to amplify a 0.8 kB region of the intact ACB1 gene, and therefore the acb1 deleted mutant would not produce the 0.8 kB band. A second set of primers was designed to produce a band only when the ACB1 gene was deleted. Specifically, one primer (i.e., 3UTR-URA3; SEQ ID NO:16) binds to a region in the vector sequences of the introduced 4.6 kB Ascl/SphI disruption fragment, and the other primer (i.e., 3R-ACBPn; SEQ ID NO:17) binds to the ACB1 terminator sequences of chromosome outside of the homologous region of the disruption fragment.

More specifically, the colony PCR was performed using a reaction mixture that contained: 20 mM Tris-HCl (pH 8.4), 50 mM KCl, 1.5 mM MgCl₂, 400 μM each of dGTP, dCTP, dATP, and dTTP, 2 μM each of the primers, 20 μl water and 2 U Taq polymerase. Amplification was carried out as follows: initial denaturation at 94° C. for 120 sec, followed by 35 cycles of denaturation at 94° C. for 60 sec, annealing at 55° C. for 60 sec, and elongation at 72° C. for 120 sec. A final elongation cycle at 72° C. for 5 min was carried out, followed by reaction termination at 4° C.

Of thirty colonies screened, 25 had the ACB1 knockout fragment integrated at a random site in the chromosome and thus were not Δacb1 mutants but could grow on ura-plates. Three of these random integrants, designated as Y4036U-1, Y4036U-2 and Y4036U-3 were used as controls in oil and lipid production experiments (infra).

The remaining 5 of 30 colonies screened contained the acb1 knockout. These five Δacb1 mutants within the Y4036U strain background were named RHY14, RHY15, RHY16, RHY17 and RHY18. Further confirmation of the acb1 knockout was performed by quantitative real time PCR on the ACB1 gene (i.e., YL ACBP), with the Yarrowia translation elongation factor (tef-1) gene (GenBank Accession No. AF054510) used as the control. Real time PCR primers and a TaqMan probe targeting the ACB1 gene and the tef-1 gene were designed with Primer Express software v. 2.0 (Applied Biosystems, Foster City, Calif.). Specifically, real time PCR primers ef-324F (SEQ ID NO:18), ef-392R (SEQ ID NO:19), ACB1-378F (SEQ ID NO:20) and ACB1-474R (SEQ ID NO:21) were designed, as well as the TaqMan probes ef-345T (i.e., 5′ 6-FAM™-TGCTGGTGGTGTTGGTGAGTT-TAMRA™, wherein the nucleotide sequence is set forth as SEQ ID NO:22) and ACB1-398T (i.e., 5′-6FAM™-ACCGACCCGGCGCCTTCA-TAMRA™, wherein the nucleotide sequence is set forth as SEQ ID NO:23). The 5′ end of the TaqMan fluorogenic probes have the 6FAM™ fluorescent reporter dye bound, while the 3′ end comprises the TAMRA™ quencher. All primers and probes were obtained from Sigma-Genosys (Woodlands, Tex.).

The knockout candidate DNA was prepared by suspending 1 colony each of RHY14, RHY15, RHY16, RHY17 and RHY18 in 50 μl of water. Reactions for tef-1 and ACB1 were run separately, in triplicate for each sample. Real time PCR reactions included 20 pmoles each of forward and reverse primers (i.e., ef-324F, ef-392R, ACB1-378F and ACB1-474R, supra), 5 pmoles TaqMan probe (i.e., ef-345T and ACB1-398T, supra), 10 μl TaqMan Universal PCR Master Mix—No AmpErase® Uracil-N-Glycosylase (UNG) (Catalog No. PN 4326614, Applied Biosystems), 1 μl colony suspension and 8.5 μl RNase/DNase free water for a total volume of 20 μl per reaction. Reactions were run on the ABI PRISM® 7900 Sequence Detection System under the following conditions: initial denaturation at 95° C. for 10 min, followed by 40 cycles of denaturation at 95° C. for 15 sec and annealing at 60° C. for 1 min. Real time data was collected automatically during each cycle by monitoring 6-FAM™ fluorescence. Data analysis was performed using tef-1 gene threshold cycle (CT) values for data normalization as per the ABI PRISM® 7900 Sequence Detection System instruction manual.

Evaluation of Total Oil Production in Yarrowia lipotytica Strain Y4036U Δacb1 Mutants

To evaluate the effect of the ACB1 gene knockout on the total lipid content in the cells and the percent of PUFAs in the total lipid fraction, the ACB1 wild type (i.e., strains Y4036U-1, Y4036U-2 and Y4036U-3 having the ACB1 knockout fragment integrated at a random site in the chromosome) and duplicate acb1 mutant strains (i.e., the Y4036U Δacb1 mutants designated as RHY15, RHY16, RHY17 and RHY18) were grown under comparable oleaginous conditions. Specifically, cultures were grown at a starting OD₆₀₀ of ˜0.1 in 25 mL of fermentation media (FM) in a 125 mL flask for 24 hrs. The cells were harvested by centrifugation for 5 min at 4300 rpm in a 50 mL conical tube. The supernatant was discarded and the cells were re-suspended in 25 mL of HGM and transferred to a new 125 mL flask. The cells were incubated with aeration for an additional 120 hrs at 30° C.

To determine the dry cell weight [“DCW”], the cells from 5 mL of the HGM-grown cultures were processed. The cultured cells were centrifuged for 5 min at 4300 rpm. The pellet was re-suspended using 10 mL of sterile water and was centrifuged under the same conditions for a second time. The pellet was then re-suspended using 1 mL of sterile water (three times) and was transferred to a pre-weighed aluminum pan. The cell suspension was dried overnight in a vacuum oven at 80° C. The weight of the cells was determined.

To determine the lipid content, 1 mL of HGM cultured cells were similarly collected by centrifugation for 1 min at 13,000 rpm, total lipids were extracted, and fatty acid methyl esters (FAMEs) were prepared by trans-esterification, and subsequently analyzed with a Hewlett-Packard 6890 GC (General Methods).

The total dry cell weight of the cells [“DCW’], the total lipid content of cells [“TFAs % DCW”], the concentration of each fatty acid as a weight percent of TFAs [“% TFAs”] and the DGLA content as a percent of the dry cell weight [“DGLA % DCW”] are shown below in Table 4, for each of the ACB1 wild type strains having the ACB1 knockout fragment integrated at a random site in the chromosome (i.e., strains Y4036U-1, Y4036U-2 and Y4036U-3) and for each of the Y4036U Δacb1 mutant strains (i.e., RHY15, RHY16 [grown in duplicate], RHY17 and RHY18 [grown in duplicate]). One of the duplicate cultures of RHY15 and one of the duplicate cultures of RHY17 produced less than 0.5 g/L DCW; thus, sufficient cell mass was not available for analysis and the cultures were not included in the results presented below. More specifically, fatty acids will be identified as 16:0 (palmitate), 16:1 (palmitoleic acid), 18:0 (stearic acid), 18:1 (oleic acid), 18:2 (LA), 20:2 (EDA), 20:3 (DGLA), 20:4 (ARA) and other.

TABLE 4
Lipid Composition of Y4036U Y. lipolytica Strains With Or Without acb1 Deletion

| | | | % TFAs | | | | | | | | | |
|---|---|---|---|---|---|---|---|---|---|---|---|---|
| | | TFAs % | 16:0 | 16:1 | 18:0 | 18:1 | 18:2 | 20:2 | 20:3 | 20;4 | | DGLA % |
| Strain | DCW | DCW | Palmitic | Palmitoleic | Stearic | Oleic | Linoleic | EDA | DGLA | ARA | Other | DCW |
| Y4036U-1 | 2.88 | 23 | 8.2 | 4.1 | 1.9 | 21.9 | 31.7 | 7.7 | 16.6 | 0.4 | 7.6 | 3.8 |
| Y4036U-2 | 4.16 | 26 | 7.7 | 2.6 | 3.3 | 24.8 | 28.3 | 10.0 | 16.4 | 0.3 | 6.5 | 4.2 |
| Y4036U-3 | 1.96 | 20 | 7.6 | 4.4 | 1.7 | 20.5 | 27.8 | 9.9 | 18.5 | 0.4 | 9.2 | 3.7 |
| Y4036U- | | 23 | | | | | | | 17.2 | | | 3.9 |
| average | | | | | | | | | | | | |
| RHY15-1 | 2.28 | 16 | 5.4 | 1.1 | 2.2 | 19.4 | 36.6 | 9.3 | 21.3 | 0.4 | 4.2 | 3.4 |
| RHY16-1 | 1.28 | 14 | 5.6 | 1.3 | 1.8 | 18.6 | 35.6 | 9.8 | 22.0 | 0.0 | 5.2 | 3.0 |
| RHY16-2 | 3.30 | 17 | 5.1 | 1.0 | 2.5 | 19.3 | 36.9 | 9.0 | 21.2 | 0.4 | 4.7 | 3.7 |
| RHY17-1 | 3.68 | 17 | 5.1 | 0.9 | 2.6 | 19.6 | 36.4 | 9.3 | 21.1 | 0.4 | 4.6 | 3.6 |
| RHY18-1 | 1.96 | 14 | 5.5 | 1.2 | 2.0 | 19.5 | 36.3 | 9.8 | 21.6 | 0.4 | 3.7 | 3.1 |
| RHY18-2 | 2.00 | 15 | 5.4 | 1.2 | 2.0 | 19.4 | 36.1 | 9.9 | 21.6 | 0.5 | 4.0 | 3.2 |
| Y4036U | | 15 | | | | | | | 21.4 | | | 3.3 |
| Δacb1- | | | | | | | | | | | | |
| average | | | | | | | | | | | | |

Table 4 shows that there was an approximately 30% decrease in total oil content (TFAs % DCW) for the chromosomal acb1 deletion in Y4036U, compared to that for the wild type ACB1 Y4036U strain. There was approximately a 20% increase in DGLA % TFAs in the acb1 mutants but the DGLA productivity (DGLA % DCW) was not significantly changed, as compared to controls.

### Example 2

### Overexpression of YL ACBP in Yarrowia lipolytica Strain Y4305U1

The present Example describes the generation of pZP2-YACBP, comprising a chimeric FBAIN::YL ACBP::PEX20 gene. This plasmid was then transformed into Yarrowia lipolytica strain Y4305U1 to determine the results of overexpression of the ACBP gene. Increased fatty acid content and modification of the relative abundance of each fatty acid species was observed.

### Generation of Plasmid PZP2-YACBP

Oligonucleotides YACBP-F (SEQ ID NO:24) and YACBP-R (SEQ ID NO:25) were designed and synthesized to allow amplification of the ACBP ORF from Yarrowia lipolytica genomic DNA (isolated from strain ATCC #20362).

The PCR reactions, with Y. lipolytica genomic DNA as template, were individually carried out in a 50 μl total volume comprising: 1 μl each of 20 μM forward and reverse primers, 1 μl genomic DNA (100 ng), 22 μl water and 25 μl 2× premix of ExTaq Taq polymerase (TaKaRa Bio Inc., Otsu, Shiga, 520-2193, Japan). Amplification was carried out at 94° C. for 1 min, followed by 30 cycles at 94° C. for 20 sec, 55° C. for 20 sec, and 72° C. for 20 sec, followed by a final elongation cycle at 72° C. for 5 min. A ˜250 bp DNA fragment was generated that contained the YL ACBP ORF. The PCR fragment was purified with a Qiagen PCR purification kit following the manufacturer's protocol. Purified DNA sample was digested with NcoI and NotI, and then purified with a Qiagen reaction clean-up kit.

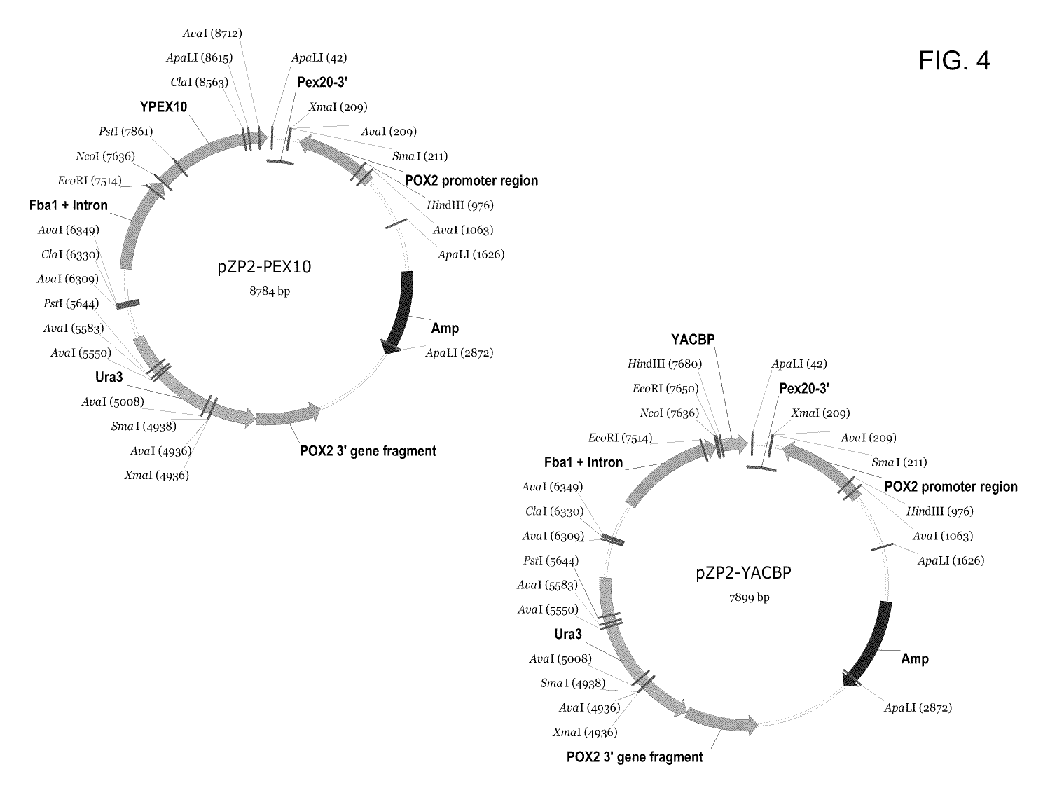

Separately, vector pZP2-PEX10 (FIG. 4A; SEQ ID NO:26) was digested with NcoI and NotI, and the 7.6 kB fragment containing the vector backbone without the Yarrowia lipolytica PEX10 gene (encoding GenBank Accession No. CAG81606) was isolated by gel electrophoresis and purified with a Qiagen Gel purification kit.

The YL ACBP fragment was directionally ligated with the pZP2-PEX10 vector (SEQ ID NO:26). Specifically, the ligation reaction contained: 10 μl 2× ligation buffer, 1 μl T4 DNA ligase (Promega), 4 μl (˜300 ng) of the 250 bp PCR fragment containing the YL ACBP ORF, and 1 μl of the 7.6 kB fragment from pZP2-PEX10 (˜150 ng). The reaction mixtures were incubated at room temperature for 2 hrs and used to transform E. coli Top10 competent cells (Invitrogen). Plasmid DNA from transformants was recovered using a Qiagen Miniprep kit. Correct clones were identified by restriction mapping and the final construct was designated “pZP2-YACBP”.

Thus, pZP2-YACBP (FIG. 4B) thereby contained the following components:

TABLE 5
Components Of Plasmid pZP2-YACBP (SEQ ID NO: 27)

| RE Sites And | |
|---|---|
| Nucleotides | |
| Within SEQ ID | Description Of Fragment And |
| NO: 27 | Chimeric Gene Components |
| BglII-BsiWI | FBAIN::YL ACBP::PEX20, comprising: |
| (6681-301) | FBAIN: Yarrowia lipolytica FBAIN promoter (U.S. |
| | Pat. No. 7,202,356); |
| | YL ACBP: Yarrowia lipolytica acb1 gene (SEQ ID |
| | NO: 1; labeled as “YACBP” in Figure;) |
| | Pex20: Pex20 terminator sequence of Yarrowia |
| | Pex20 gene (GenBank Accession No. AF054613) |
| (4494-5981) | Yarrowia URA3 (GenBank Accession No. AJ306421) |
| ApaI-PacI | 3′-noncoding region of Yarrowia Pox2 gene |
| (3836-4493) | (GenBank Accession No. AJ001300) |
| (2116-2976) | ampicillin-resistance gene (Amp^{R}) for selection |
| | in E. coli |
| (318-1127) | 5′-noncoding region of Yarrowia Pox2 gene |
| | (GenBank Accession No. AJ001300) |

Functional Analysis of Yarrowia lipolytica Strain Y4305U1 Transformants Overexpressing YL ACBP

A clone of ZP2-YACBP was transformed into Yarrowia-lipolytica strain Y4305U1, as described in the General Methods (non-transformed cells of Yarrowia lipolytica strain Y4305 served as the control). The transformants were selected onto MM plates.

The cells from each transformation were plated onto MM plates and maintained at 30° C. for 2 days. Three transformants from each transformation plate were used to inoculate individual 25 mL cultures with MM medium. Each culture was allowed to grow for 2 days at 30° C., then switched into 25 mL of HGM and allowed to grow for 5 days at 30° C.

The cells were collected by centrifugation, total lipids were extracted, and fatty acid methyl esters [“FAMEs”] were prepared by trans-esterification, and subsequently analyzed with a Hewlett-Packard 6890 GC.

Based on the above analyses, lipid content and composition was determined in one transformant (i.e. #6) of Y4305U1 transformed with pZP2-YACBP, and the control strain of Y4305 respectively, as shown below in Table 6. Two cultures of each strain were analyzed, while the average results are shown in the rows highlighted in grey.

Specifically, the total lipid content of cells [“TFAs % DCW”], the concentration of EPA as a weight percent of TFAs [“EPA % TFAs”] and the EPA content as a percent of the dry cell weight [“EPA % DCW”] and compared in Table 6.

DCW was determined by collecting cells from 10 mL of culture via centrifugation, washing the cells with water once to remove residue medium, drying the cells in a vacuum oven at 80° C. overnight, and weighing the dried cells.

TABLE 6
Lipid Content And Composition In Yarrowia
Strain Y4305 Overexpressing YL ACBP

| | | TFAs % | EPA % | EPA % |
|---|---|---|---|---|
| Sample | Strain | DCW | TFAs | DCW |
| 1 | Y4305 | 29.61 | 52.13 | 15.44 |
| 2 | Y4305 | 29.75 | 52.51 | 15.62 |
| Avg. | | 29.68 | 52.32 | 15.53 |
| 3 | Transformant #6 of Y4305U1 + | 32.02 | 50.23 | 16.08 |
| | pZP2-YACBP | | | |
| 4 | Transformant #6 of Y4305U1 + | 32.22 | 50.43 | 16.25 |
| | pZP2-YACBP | | | |
| Avg. | | 32.12 | 50.33 | 16.17 |

GC analyses showed that there was an ˜8.2% increase in TFAs % DCW in Y4305U1 cells carrying pZP2-YACBP, as compared to the control cells. Furthermore, there was also an increase in EPA % DCW.

### Example 3

### Co-Expression of YL ACBP With YL DGAT In Yarrowia lipolytica Strain Y4305U

The present example describes co-expression of the Yarrowia acyl-CoA binding protein homolog (YL ACBP) with either plasmid pFBAIN-YDGAT1 (comprising the Y. lipolytica DGAT10RF) or plasmid pFBAIN-YDGAT2 (comprising the Y. lipolytica DGAT2 ORF).

Vectors pFBAIN-YDGAT1 (SEQ ID NO:33) and pFBAIN-YDGAT2 (SEQ ID NO:34), comprising a chimeric FBAINm::YL DGAT1::PEX20 gene and a chimeric FBAINm::YL DGAT2::PEX20 gene, respectively, are described in International Publication No. WO 2008/147935.

In order to disrupt the Ura3 gene in transformant #6 of Y4305U1+pZP2-YACBP (Example 2), construct pZKUM (SEQ ID NO:35; described in Table 15 of U.S. Pat. Appl. Pub. No. 2009-0093543-A1) will be used to integrate a Ura3 mutant gene into the Ura3 gene of transformant #6 of Y4305U1+pZP2-YACBP. Transformants will be grown on MM+5-FOA plates, picked and re-streaked onto MM plates and MM+5-FOA plates, separately. Strains having a Ura-phenotype (i.e., cells could grow on MM+5-FOA plates, but not on MM plates) will be selected.

Those strains having a Ura-phenotype, which are derived from Y. lipolytica strain Y4305U1 and are over-expressing YL ACBP, will then be transformed with one of the following: plasmid pFBAIn-YDGAT1 (SEQ ID NO:33; comprising the Y. lipolytica DGAT10RF), plasmid pFBAIN-YDGAT2 (SEQ ID NO:34; comprising the Y. lipolytica DGAT2 ORF), or pFBAIn-MOD-1 (SEQ ID NO:32; a “control” vector).

Transformants will be grown in FM medium for 2 days, followed by HGM medium for 5 days. The cells will be collected by centrifugation, and lipids will be extracted. Fatty acid methyl esters will be prepared by trans-esterification and subsequently analyzed with a Hewlett-Packard 6890 GC.

Since DGAT1 and DGAT2 catalyze the last step of TAG biosynthesis (i.e., the incorporation of acyl-CoA into DAG to form TAG with the release of the CoA moiety) and since YL ACBP can bind to acyl-CoA molecules and increase their intracellular concentration, co-expression of YL ACBP and either YL DGAT1 or YL DGAT2 is expected to lead to a synergistic effect that will be greater than that achieved by overexpression of either ACBP or DGAT.

What is claimed is:

1. An oleaginous microbial organism, comprising:
(i) a recombinant construct comprising at least one isolated polynucleotide comprising a nucleic acid sequence encoding an acyl-CoA binding protein operably linked to at least one regulatory sequence; and
(ii) a source of fatty acids.

2. An oleaginous microbial organism, comprising:
(i) a disruption in a native gene encoding an acyl-CoA binding protein; and
(ii) a source of fatty acids.

3. The oleaginous microbial organism of either claim 1 or claim 2, selected from the group consisting of Yarrowia, Candida, Rhodotorula, Rhodosporidium, Cryptococcus, Trichosporon and Lipomyces.

4. The oleaginous microbial organism of claim 3 wherein the organism accumulates at least about 25% of its dry cell weight as oil.

5. The oleaginous microbial organism of claim 3 wherein the organism is Yarrowia lipolytica.

6. The oleaginous microbial organism of claim 1 comprising a recombinant construct having at least one nucleic acid sequence encoding a diacylglycerol acyltransferase selected from the group consisting of DGAT1, DGAT2 and DGAT1 in combination with DGAT2.

7. A method for modifying total lipid content in an oleaginous microbial organism, comprising:
a) providing an oleaginous microbial organism, comprising:
(i) a recombinant construct comprising at least one isolated polynucleotide comprising a nucleic acid sequence encoding an acyl-CoA binding protein operably linked to at least one regulatory sequence; and
(ii) a source of fatty acids;
b) growing the cell of step (a) under conditions whereby transfer of the fatty acids to lipid fractions of the organism is altered by altering expression of the isolated polynucleotide comprising a nucleic acid sequence encoding an acyl-CoA binding protein; and
c) optionally recovering the total lipid fractions of step (b).

8. The method of claim 7 wherein the alteration is overexpression of acyl-CoA binding protein.

9. The method of claim 7 wherein the alteration is disruption of acyl-CoA binding protein.

10. The method of claim 7 wherein oleaginous microbial organism further comprises a recombinant construct having at least one nucleic acid sequence encoding a diacylglycerol acyltransferase selected from the group consisting of DGAT1, DGAT2 and DGAT1 in combination with DGAT2.

11. The method of claim 7, wherein the oleaginous yeast is selected from the group consisting of: Yarrowia, Candida, Rhodotorula, Rhodosporidium, Cryptococcus, Trichosporon and Lipomyces.

12. A method for modifying fatty acid composition in an oleaginous microbial organism, comprising:
(a) providing an oleaginous microbial organism, comprising:
(i) a recombinant construct comprising at least one isolated polynucleotide comprising a nucleic acid sequence encoding an acyl-CoA binding protein operably linked to at least one regulatory sequence; and
(ii) a functional ω-3/ω-6 fatty acid biosynthetic pathway comprising at least one isolated polynucleotide comprising a nucleic acid sequence encoding a desaturase enzyme for biosynthesis of fatty acids;
(b) growing the cell of step (a) under conditions whereby the expression of the acyl-CoA binding protein is altered, resulting in an altered rate of desaturation by the at least one desaturase enzyme and an altered transfer of the synthesized fatty acids to lipid fractions of the organism;
(c) optionally recovering the lipid fractions of step (b); and,
(d) optionally determining the modified fatty acid composition of the lipid fractions of step (c).

13. The method of claim 12, wherein expression of the at least one isolated polynucleotide comprising a nucleic acid sequence encoding an acyl-CoA binding protein is decreased, thereby resulting in an increased rate of desaturation by the at least one desaturase enzyme, increased production of polyunsaturated fatty acids as a percent of the total fatty acids, and decreased total lipid content in the oleaginous microbial organism.

14. The method of claim 12, wherein the expression of the at least one isolated polynucleotide comprising a nucleic acid sequence encoding an acyl-CoA binding protein is increased, thereby resulting in a decreased rate of desaturation by the at least one desaturase enzyme, decreased production of polyunsaturated fatty acids as a percent of the total fatty acids, and increased total lipid content in the oleaginous microbial organism.

15. The method of either claim 7 or claim 12, wherein said acyl-CoA binding protein comprises the following amino acid sequence motifs: SEQ ID NO:36, SEQ ID NO:37 and SEQ ID NO:38.

16. A method for increasing the total lipid content in a Yarrowia sp., comprising:
a) providing Yarrowia sp., comprising:
i) a recombinant construct comprising at least one isolated polynucleotide comprising a nucleic acid sequence encoding an acyl-CoA binding protein, said protein selected from the group consisting of:
(a) a protein consisting essentially of the sequence set forth in SEQ ID NO:2; and
(b) a protein comprising the following amino acid sequence motifs: SEQ ID NO:36, SEQ ID NO:37 and SEQ ID NO:38;
wherein said isolated polynucleotide is operably linked to at least one regulatory sequence; and
b) a source of fatty acids;
c) growing the Yarrowia sp. of step (a) under conditions whereby the expression of the acyl-CoA binding protein results in an increased total lipid content of at least 5% when compared to the total lipid content of a Yarrowia sp. lacking said recombinant construct; and,
d) optionally recovering the total lipids of step (b).

17. The method of claim 16, wherein the fatty acids of step (b) are endogenously produced by expression of a functional ω-3/ω-6 fatty acid biosynthetic pathway.

18. The method of claim 17, wherein said fatty acids comprise at least one polyunsaturated fatty acid selected from the group consisting of: linoleic acid, conjugated linoleic acid, γ-linolenic acid, dihomo-γ-linolenic acid, arachidonic acid, α-linolenic acid, stearidonic acid, eicosatetraenoic acid, eicosapentaenoic acid, ω-6 docosapentaenoic acid, ω-3 docosapentaenoic acid, eicosadienoic acid, eicosatrienoic acid, docosatetraenoic acid, docosahexaenoic acid, and hydroxylated or expoxy fatty acids thereof.

19. The method of claim 16 wherein said Yarrowia sp. comprises a recombinant construct having at least one DAG AT sequence selected from the group consisting of:
a) a DGAT1 enzyme consisting essentially of the sequence set forth in SEQ ID NO:4;
b) a DGAT2 enzyme consisting essentially of a sequence selected from the group consisting of SEQ ID NO:6, SEQ ID NO:8, SEQ ID NO:9, SEQ ID NO:10 and SEQ ID NO:11; and,
c) a DGAT1 as described in part (a) in combination with DGAT2 as described in part (b).
